SEQUENCE LISTING (14 sequences)

SEQ ID NO: 1 - protein, 237 aa, synthetic construct
  REGION 1..237: note = Synthetic antigen (sfGFP-D117C)
SKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGCTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYK

SEQ ID NO: 2 - protein, 273 aa, synthetic construct
  REGION 1..273: note = Synthetic antigen (sfGFP-PIGF)
SKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGCTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKGGGGSGGGGSGGGGRRRPKGRGKRRREKQRPTDCHL

SEQ ID NO: 3 - protein, 82 aa, synthetic construct
  REGION 1..82: note = Synthetic Antigen (ALFA-PIGF)
MPSRLEEELRRRLTEPPSRLEEELRRRLTEPPSRLEEELRRRLTEGGGGSGGGGSGGGGSRRRPKGRGKRRREKQRPTDCHL

SEQ ID NO: 4 - protein, 343 aa, synthetic construct
  REGION 1..343: note = GFP CAR
MLLLVTSLLLCELPHPAFLLIPQVQLQESGGGSVQAGGSLKLSCAASGGAYRNACMGWFRQAPGKEREGVAIINSVDTTYYADPVKGRFTISRDNAKSTVYLLMNSLKPEDTAIYYCAQVARVVCPGDKLGASGYNYWGQGTQVTVSSGSESKYGPPCPCPAFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 339 aa, synthetic construct
  REGION 1..339: note = ALFA CAR
MLLLVTSLLLCELPHPAFLLIPEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGSESKYGPPCPCPAFWVLVVVGGVLACYSLLVTVAFIIFWVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 232 aa, synthetic construct
  REGION 1..232: note = mScarlet reporter
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 7 - protein, 19 aa, synthetic construct
  REGION 1..19: note = T2A peptide
EGRGSLLTCGDVEENPGPG

SEQ ID NO: 8 - DNA, 1178 bp, synthetic construct
  misc_feature 1..1178: note = EF1a promoter
  source 1..1178: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 9 - protein, 104 aa, Homo sapiens
MTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 10 - protein, 90 aa, Homo sapiens
MNEGSVTGSCYCGKRISSDSPPSVQFMNRLRKHLRAYHRCLYYTRFQLLSWSVCGGNKDPWVQELMSCLDLKECGHAYSGIVAHQKHLLP

SEQ ID NO: 11 - protein, 90 aa, synthetic construct
  REGION 1..90: note = hCXCL16K42A
MNEGSVTGSCYCGARISSDSPPSVQFMNRLRKHLRAYHRCLYYTRFQLLSWSVCGGNKDPWVQELMSCLDLKECGHAYSGIVAHQKHLLP

SEQ ID NO: 12 - protein, 88 aa, Mus musculus
NQGSVAGSCSCDRTISSGTQIPQGTLDHIRKYLKAFHRCPFFIRFQLQSKSVCGGSQDQWVRELVDCFERKECGTGHGKSFHHQKHLP

SEQ ID NO: 13 - protein, 90 aa, synthetic construct
  REGION 1..90: note = hCXCL16R73A
MNEGSVTGSCYCGKRISSDSPPSVQFMNRLRKHLRAYHRCLYYTAFQLLSWSVCGGNKDPWVQELMSCLDLKECGHAYSGIVAHQKHLLP

SEQ ID NO: 14 - protein, 71 aa, Homo sapiens
MASNYDCCLSYIQTPLPSRAIVGFTRQMADEACDINAIIFHTKKRKSVCADPKQNWVKRAVNLLSLRVKKM

(19) United States
(12) Patent Application Publication
(10) Pub. No.: US 2024/0016847 A1
(43) Pub. Date: Jan. 18, 2024

(54) Probiotic-Guided CAR-T Cells for Tumor Targeting

(71) Applicant: The Trustees of Columbia University in the City of New York, New York, NY (US)

(72) Inventors: Tal DANINO, Brooklyn, NY (US); Nicholas ARPAIA, New York, NY (US); Rosa VINCENT, New York, NY (US); Thomas SAVAGE, Bronx, NY (US); Jongwon IM, New York, NY (US); Candice GURBATRI, Dix Hills, NY (US)

(73) Assignee: The Trustees of Columbia University in the City of New York, New York, NY (US)

(21) Appl. No.: 18/451,576

(22) Filed: Aug. 17, 2023

Related U.S. Application Data
(63) Continuation of application No. PCT/US22/16775, filed on Feb. 17, 2022.
(60) Provisional application No. 63/150,191, filed on Feb. 17, 2021.
(60) Provisional application No. 63/254,305, filed on Oct. 11, 2021.

(51) Int. Cl. A61K 35/17; A61K 35/74; C12N 1/20; C12N 5/0783; C12N 15/10

(52) U.S. Cl. CPC A61K 35/17; A61K 35/74; C12N 1/205; C12N 5/0636; C12N 15/102

(57) ABSTRACT

A system combining programmable bacteria cells that produce one or more antigens and optionally one or more cytokines and CAR-T cells that recognize and respond to at least one of the antigens to elicit an immune response against tumors and treat hyperproliferative disorders.

### CROSS-REFERENCE TO RELATED APPLICATIONS

Priority is claimed to U.S. Provisional Applications Nos. 63/150,191 filed 17 Feb. 2021 and 63/254,305 filed on 11 Oct. 2021, each of which is hereby incorporated by reference in its entirety.

### STATEMENT REGARDING FEDERALLY SPONSORED RESEARCH OR DEVELOPMENT

This invention was made with government support under R01EB030352 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on 13 Feb. 2022, is named SEQL.txt and is 20,819 bytes in size.

### TECHNICAL FIELD OF THE INVENTION

This disclosure generally relates to the fields of medicine and immunology. More specifically, the disclosure relates to compositions capable of programmed delivery of synthetic antigens that interact with tumor cells and chimeric antigen receptor (CAR) T cells that recognize those synthetic antigens. Programmed delivery of additional molecules such as chemokines that facilitate recruitment of the CAR-T cells to the synthetic antigens are also contemplated herein.

### BACKGROUND OF THE INVENTION

Recent advances in synthetic biology have united with cancer immunotherapy to refocus the immunosurveillance searchlight in the form of intelligent, tumor-antigen targeting therapies like chimeric antigen receptor (CAR)-T cells. The central challenge of antigen-targeted cell therapies is intrinsically tied to the expression patterns of the antigen itself, making the identification of an optimal target one of the most critical factors in the development of new CARs. While expression patterns are a consideration, they are not the only consideration; for example, even the gold standard CD19 CAR faces antigen-dependent issues, the loss of which has become a frequent cause of patient relapse, and off-tumor expression on brain mural cells has been linked to reports of dangerous neurotoxicity.

Antigen-related challenges play an even larger role in treating solid tumors, and while there has been success in the use of CAR-T cells for hematological malignancies, translation to solid tumors has been greatly limited. Strategies targeting more than one antigen circumvent issues of tumor escape; however, the challenge of finding a single suitable target has limited this approach for most solid tumors. Few TAAs identified on solid tumors are tumor-specific, and carry a high risk of fatal on-target, off-tumor toxicity due to cross-reactivity against vital tissues. Moreover, if a safe target can be identified, TAAs are often heterogeneously expressed and selection pressures from targeted therapies ultimately lead to antigen-loss and tumor escape. Strategies targeting more than one antigen circumvent issues of tumor escape; however, the challenge of finding a single suitable target has limited this approach for most solid tumors.

While emerging strategies to address the antigen bottleneck have focused on improving CAR-T cells with additional genetic circuitry modulatory proteins and combinations with nanoparticles and oncolytic viruses, new approaches are clearly needed.

### BRIEF SUMMARY OF THE INVENTION

The present disclosure relates to a platform or system for activating an immune response against tumor cells to thereby treat hyperproliferative disorders. The components of the system comprise programmable bacteria cells that produce one or more synthetic antigens and immune T cells expressing a chimeric antigen receptor (CAR-T cell), wherein the CAR-T cells are engineered to recognize and respond to the antigen and activate an immune response against tumor cells. In some embodiments, the programmable bacteria cells produce one or more synthetic antigens and one or more cytokines. In some embodiments, the system includes a first strain of programmable bacteria cells that produce the one or more synthetic antigens and a second strain of programmable bacterial cells that produce one or more cytokines.

Programmable bacterial cells described herein comprise a nucleic acid encoding the one or more synthetic antigens and/or one or more chemokines operably linked to at least one transcriptional element, wherein at least one transcriptional element modulates the expression or secretion of the one or more synthetic antigens and/or one or more cytokines in response to the density or quantity of the programmable bacterial cells in vitro or in vivo (e.g., in a tumor microenvironment). In some embodiments, the transcriptional element is a synchronized lysis circuit comprising a nucleic acid encoding a quorum-sensing gene, a nucleic acid encoding a lysis gene, a promoter, and a terminator contained on a single operon.

In some embodiments, the synthetic antigens are green fluorescent proteins (GFP). In particular embodiments, the GFP are super-folder green fluorescent proteins (sfGFP). In one particular embodiment, the sfGFP is a dimeric version of sfGFP that comprises a 20-amino acid peptide-tag derived from the heparin binding domain of placenta growth factor-2 (sfGFP^{PlGF}) In another particular embodiment, the sfGFP is a soluble dimer comprising a cystine substitution at position D117 (sfGFP^{D117C}). In some embodiments, the synthetic antigen comprises an ALFA-tag linked to 20-amino acid peptide-tag PlGF derived from the heparin binding domain of placenta growth factor-2 (ALFA^{PlGF}).

In some embodiments, the cytokine is IL-12. In some embodiments, the one or more cytokines include a chemokine. In some embodiments, the chemokine is a member of the CXC family of chemokines. In some embodiments, the chemokine is CXCL9. In other embodiments, chemokine is CXCL16. In some embodiments, the chemokine is an activating form of CXCL16, e.g., CXCL16^{K42A}. In some embodiments, the chemokine is CCL20.

In some embodiments, the transcriptional element in the programmable bacterial cells modulate the expression or secretion of the one or more synthetic antigens and the one or more cytokines. In other embodiments, a different transcriptional element modulates the expression or secretion of the one or more synthetic antigens and the one or more cytokines.

In some embodiments, the programmable bacterial cells belong to at least one genus selected from the group consisting of Salmonella, Escherichia, Firmicutes, Bacteroidetes, Lactobacillus, and Bifidobacteria. In some embodiments, the programmable bacterial cells belong to the genus Escherichia. In particular embodiments, the programmable bacterial cells are Escherichia coli Nissle (EcN) cells. In one embodiment, the EcN cells comprise a knockout of the FliC gene. In one embodiment, the EcN cells comprise a knockout of the msbB gene. In one embodiment, the EcN cells comprise a knockout of the FliC gene and the msbB gene.

In some embodiments, the CAR-T cells are designed to target the GFP antigens. In some embodiments, the CAR-T cells are designed to target the sfGFP antigens. In some embodiments, the CAR-T cells are designed to target the ALFA-tag antigens. In some embodiments, the CAR-T cells are designed to chemotactically respond to cytokines released or otherwise produced by the programmable bacterial cells.

The present disclosure also relates to methods of treating a hyperproliferative disorder comprising administering to a subject the system of programmable bacterial and CAR-T cells described herein. In some embodiments, the hyperproliferative disorder is selected from the group consisting of breast cancer, melanoma, renal cancer, prostate cancer, pancreatic adenocarcinoma, colorectal cancer, lung cancer, esophageal cancer, squamous cell carcinoma of the head and neck, liver cancer, ovarian cancer, cervical cancer, thyroid cancer, glioblastoma, and glioma.

The present disclosure also relates to methods of reducing the rate of proliferation of a tumor cell comprising administering to a subject the system of programmable bacterial and CAR-T cells described herein. The present disclosure also relates to methods of killing a tumor cell comprising administering to a subject the system of programmable bacterial and CAR-T cells described herein.

In some embodiments, the programmable bacterial cells and/or CAR-T cells may be administered to a subject or delivered to a tumor in the form of a pharmaceutical composition, which may comprise one or more pharmaceutically acceptable carriers, diluents, or excipients.

The present disclosure also relates to articles of manufacture useful for treating a hyperproliferative disorder. In some embodiments, the articles of manufacture comprise a container comprising programmable bacterial cells described herein, or pharmaceutical compositions comprising the same, as well as instructional materials for using the same to treat a hyperproliferative disorder. In some embodiments, the articles of manufacture are part of a kit that comprises a bacterial culture vessel and/or bacterial cell growth media. In some embodiments, the articles of manufacture comprise a container comprising CAR-T cells described herein, or pharmaceutical compositions comprising the same, as well as instructional materials for using the same to treat a hyperproliferative disorder.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description of the Invention. This summary is not intended to identify key features or essential features of the claimed subject matter, nor is it intended to be used as an aid in determining the scope of the claimed subject matter.

### BRIEF DESCRIPTION OF THE SEVERAL VIEWS OF THE DRAWINGS

FIG. 1 shows that recombinantly produced, His-tag purified PlGF binds to collagen coated plates and strongly activates GFP-CAR⁺ Jurkats. (A) Plasmid map of protein expression vector transformed into eNiCo21(DE3) E. coli cells for protein purification of D117C and PlGF sfGFP variants. (B) Eluants of His-tag purified sfGFP variants, sizes show monomeric and dimeric molecules. (C) D117C and PlGF variants were plated in half log dilutions in PBS on collagen coated plates, incubated for 30 m at 37° C. and washed 2× with PBS to dislodge any unbound protein. Fluorescence intensity was read at 488 nm on a standard Tecan plate reader. (D) CD69 expression on GFP CAR⁺ or CD19 CAR⁺ Jurkat cells. Jurkats were plated on collagen coated plates and assessed for CD69 expression by flow cytometry following 16-hour incubation with purified GFP antigens or co-culture with CD19⁺ target cells (Nalm6). Error bars represent s.d. of biological replicates.

FIG. 2 shows that probiotic-guided CAR-T cells (ProCARs) are programmed to sense and respond to synthetic antigens (SA) released by tumor-colonizing probiotics. (A) Schematic of the ProCAR system whereby engineered E. coli Nissle 1917 specifically colonize the solid tumor core and release synthetic antigens (SA) through quorum-regulated lysis. SAs are designed with sfGFP and PlGF sequences to anchor to components of the extracellular matrix (ECM) and locally activate GFP-specific ProCAR-T cells within the TME. (B) Representative flow cytometry histograms demonstrating GFPCAR (GFP28z) surface expression and binding to purified monomeric-sfGFP (left) in primary human T cells together with the co-expression of the fluorescent marker, mScarlet (right). GFP28z contains an extracellular sfGFP-binding nanobody linked to CD28 and CD3ζ intracellular domains through a short IgG4 hinge and CD28 transmembrane domain, and co-expression of mScarlet is achieved through separation by a T2A element. (C) Flow cytometric quantification of CD69 surface expression on GFP28z exposed to collagen-bound PlGF relative to monomeric and dimeric (D117C) sfGFP controls.

FIG. 3 shows that GFP-directed CAR-T cells (GFP28z) activate, and mediate killing of target cells in response to collagen-bound sfGFP. (A) Representative flow cytometry histograms of CD25 surface expression on GFP28z following 16 hr incubation with 0.1 μg/mL of purified SA variants (D117C or PlGF), or a PBS vehicle, on collagen coated plates. (B) Representative flow cytometry plots of intracellular levels of IFNγ and TNFα in CD8⁺ GFP28z, treated as in (A). (C) SA-driven fold expansion of GFP28z. On day 14 post-activation, GFP28z T cells were washed of IL-2 and exposed to 0.1 ug/ml of D117C, PlGF, or PBS vehicle. Cells were counted every 3-4 days for 14 days post stimulation. (D,E) Overnight killing assays against ffLuc⁺ HEK293T (D) and ffLuc⁺ Nalm6 (E) target cells at defined effector to target ratios. CAR-T cells were co-cultured with target cells on collagen coated plates+/−0.1 μg/mL sfGFP-PlGF for 20 hours before lysis and addition of luciferin. Luminescence (RLU) was detected with a Tecan plate reader. Specific lysis (%) was determined by normalizing RLU to cocultures with untransduced T cells. (F) Representative flow cytometry histograms of CAR⁺CD8+ T cell degranulation 16-hours post incubation with D117C or PlGF as in (A), and staining for CD107a surface expression. (G) Quantification of cytokine levels in cell culture supernatants from GFP28z exposed to PBS (Veh.), D117C, or PlGF for 24 hr. Error bars represent s.d. of biological replicates, * p<0.05, ** p<0.01,**** p<0.0001 2-way ANOVA (D,E) or 1-way ANOVA (G), Holm-Sidak multiple comparison correction. ns, not significant; RLU, relative luminescence units.

FIG. 4 shows that GFP-directed CAR-T cells activate in response to soluble and collagen-bound sfGFP. (A) Quantification of flow cytometric analysis of intracellular staining for pro-inflammatory cytokines in response to 0.1 ng/mL soluble D117C, or collagen bound PlGF. (B) Quantification of cytokine production as measured in cell culture supernatants from GFP28z exposed to a PBS vehicle, D117C, or collagen-bound PlGF for 24 hr. Error bars represent s.d. of biological replicates, **** p<0.0001 2way (A) or 1way (B) ANOVA, Holm-Sidak multiple comparison correction.

FIG. 5 shows that the PlGF-based ProCAR system drives localized anti-tumor activity of GFP28z in a subcutaneous xenograft model of human leukemia. (A-D) Nalm6 cells (5×10⁵) were implanted subcutaneously (s.c.) into the hind flank of NSG mice. When tumor volumes reached ˜100 mm³, mice were intratumorally (I.T.) injected with 1×10⁵ CFU of engineered probiotic strains (Pro^{x}) producing D117C (Pro^{D117C}) and PlGF (Pro^{PlGF}) SA variants, or empty Pro controls (Pro⁻). 2.5×10⁶ GFP28z ProCAR-T cells were then I.T. delivered 48-hours post bacterial injection, with tumor growth monitored by caliper measurements and body weight recorded every 3-4 days (n>4 per group). Mean absolute tumor trajectories (B), survival curves (C), and mouse body weights at day 14 post bacteria treatment (D) are shown. (E) Biodistribution of Pro^{x} found in liver, spleen, and tumor samples calculated as colony-forming units (CFU) per gram of tumor. At day 3 (n=5) and day 14 (n=4) post bacteria treatment, tumor, liver, and spleen were homogenized and plated on LB agar plates containing the appropriate antibiotics for bacteria colony quantification. (F,G) ELISA quantification of sfGFP levels from tumor homogenates (F) and serum (G) taken 14 days post treatment. Error bars represent s.e.m. of biological replicates, *p<0.05, **p<0.01 ***p<0.001, ****p<0.0001, 2-way ANOVA (B) or 1-way ANOVA (D,F,G), with Holm-Sidak multiple comparison correction. Survival curve (C) ****p<0.0001, log-rank test.

FIG. 6 shows the individual growth trajectories of human tumors treated with the ProCAR system. (A) Nalm6 tumors were established in NSG mice and treated as in FIG. 5A. Individual tumor trajectories are shown. (B) Raji lymphoma cells (5×10⁵) were implanted subcutaneously into the hind flank of NSG mice. When tumor volumes reached ˜100 mm³, mice were intratumorally (I.T.) injected with 1×10⁵ CFU of Pro⁻, Pro^{D117C}, or Pro^{PlGF} strains. 2.5×10⁶ GFP28z ProCAR-T cells were then I.T. delivered 48-hours post bacterial injection. Tumor growth was monitored by caliper measurements every 3-4 days, individual tumor trajectories are shown. CFU, colony forming units

FIG. 7 shows that probiotic EcN remains localized to tumors in immunocompromised NSG mice. (A) IVIS images showing bioluminescent Pro populations over time following intratumoral injection of Raji tumors subcutaneously established as in FIG. 6. (B) At day 14 post treatment, Pro^{PlGF}-treated tumors were homogenized and plated on LB agar plates containing the appropriate antibiotics (+/−kanamycin) for bacteria colony quantification. Error bars represent s.d. of biological replicates, Student's t test; ns, not significant.

FIG. 8 shows that engineered Pro^{x} strains of E. coli Nissle 1917 (EcN) enhance T cell effector function. (A) Quantification of flow cytometric analysis assessing surface expression of CD25, CD69, and CD107a in response to EcN lysate+/−PlGF. GFP28z cells were plated on collagen-coated plates and exposed to lysate at a final OD of 1, +/−0.1 ng/mL purified PlGF. (B) Phenotype of T-cells exposed to stimulants as in (A), for 48 hr. Pie charts representing the clockwise differentiation of CD8+ T cell populations from stem cell memory (T_{scm}) CD62L⁺CD45RO⁻, to central memory (T_{cm}) CD62L⁺CD45RO⁺, to effector memory (Tₑₘ) CD62L⁻CD45RO⁺, and to terminal effector (T_{eff}) CD62L⁻CD45RO⁻ cells. (C) Quantification of cytokine levels detected in cell culture supernatants from GFP28z treated as in (A). (D-I) Nalm6 cells (5×10⁵) were implanted subcutaneously into the hind flank of NSG mice. When tumor volumes reached ˜100 mm³, mice

FIG. 9 shows that E. coli Nissle (EcN) lysate drives T cell effector phenotype. Representative flow cytometry contour plots assessing the phenotype of T cells following stimulation with either media alone or EcN lysate+/−0.1 μg/mL PlGF for 48 hr. CD8⁺ T cell populations were stained for CD45RO and CD62L expression to determine effector T cell differentiation, from stem cell memory (T_{scm}) CD62L⁺CD45RO, central memory (T_{cm}) CD62L⁺CD45RO⁺, effector memory (Tₑₘ) CD62L⁻CD45RO⁺, and terminal effector (T_{eff}) CD62LCD45RO.

FIG. 10 shows the effect of EcN strains on GFP28z cells in vivo. Nalm6 cells (5×10⁵) were implanted subcutaneously into the hind flank of NSG mice. When tumor volumes reached ˜100 mm³, mice were I.T. injected with either PBS or 1×10⁵ CFU of Pro^{PlGF} or Pro⁻. On day 2 post Pro^{x} injection, all groups received an I.T. injection of 2.5×10⁶ GFP28z ProCAR-T cells. Tumors were harvested and homogenized on day 4 for analysis. (A) Absolute counts of hCD45⁺CD3⁺ cells per mg of tumor. (B) Flow cytometric quantification of CD25 surface expression on intratumoral hCD45⁺CD3⁺CD8⁺ or CD4⁺ cells from each treatment group. (C) Quantification of cytokine levels from tumor homogenates. Error bars represent s.d. of biological replicates, * p<0.05,** p<0.01 1way (A) or 2way ANOVA (B), Holm-Sidak multiple comparison correction.

FIG. 11 shows that the ProCAR system drives durable anti-tumor effects in a subcutaneous xenograft model of triple negative breast cancer (TNBC). (A-E) MDA-MB-468 cells (5×10⁵) were implanted subcutaneously (s.c.) into the hind flank of NSG mice. When tumor volumes reached ˜50 mm³, mice were I.T. injected with either PBS or 1×10⁵ CFU of Pro^{PlGF} or empty Pro controls. Two days post injection, mice received a single I.T. injection of either PBS, 2.5×10⁶ GFP28z ProCAR-T cells, or 2.5×10⁶ ICAM1-specific CAR-T cells (ICAM28z), with tumor growth monitored by caliper measurements every 3-4 days (n>4 per group). Mean tumor trajectories (B) are shown. (C-E) Tumors treated with PBS, Pro⁻, and Pro^{PlGF} in combination with GFP28z were harvested and prepared for flow cytometry on day 30 post bacteria treatment (D55 post tumor engraftment). (C) Absolute counts of hCD45⁺CD3⁺ cells per mg of tumor. (D) Absolute counts of hCD45⁺CD3⁺CAR⁺ cells per mg of tumor. (E) Frequency of intratumoral CD4⁺ and CD8⁺ T cells displaying exhaustion markers, LAG-3, TIM-3, or PD-1, within each treatment group. (F-G) s.c. MDA-MB-468 tumors were established in NSG mice prior to I.T. injection with PBS, Pro, or Pro^{PlGF} as in (A). Mice then received an initial I.T. injection of 2.5×10⁶ untransduced (UT), or 2.5×10⁶ GFP28z ProCAR-T cells two days post bacteria treatment, followed by a second I.T. dose of UT or GFP28z 14 days later. Tumor growth was monitored by caliper measurements every 3-4 days (n>4 per group). Mean tumor trajectories are shown (G) Error bars represent s.e.m. of biological replicates, *p<0.05, **p<0.01 ***p<0.001, ****p<0.0001, 2-way (B, G) or 1-way ANOVA (C,D), with Holm-Sidak multiple comparison correction.

FIG. 12 shows T cell exhaustion in a triple negative breast cancer (TNBC) model. (A) Subcutaneous MDA-MB-468 tumors were established in NSG mice prior to I.T. injection with PBS, Pro⁻, or Pro^{PlGF} on day 26 post tumor engraftment. On day 28, mice received a single I.T. injection of either PBS, 2.5×10⁶ GFP28z ProCAR-T cells, or 2.5×10⁶ ICAM1-specific CAR-T cells (1CAM28z), as in FIG. 11A. Tumor growth was monitored by caliper measurements every 3-4 days, individual tumor trajectories are shown. (B) On day 55 tumors were taken from mice treated with PBS, Pro⁻, or Pro^{PlGF} strains in combination with GFP28z and weighed ex vivo. (C) Frequency of intratumoral hCD45+CD3+CD8+ cells displaying a terminally differentiated effector phenotype (T_{eff}, CD62L⁻CD45RO⁻). (D) Pro^{x} strains were isolated from day 55 tumor homogenates and grown overnight on the plate reader to measure OD₆₀₀ and GFP fluorescence intensity. Error bars represent s.d. of biological replicates, * p<0.05 1way ANOVA; ns, not significant.

FIG. 13 shows the individual growth trajectories of MDA-MB-468 subcutaneous TNBC tumors treated with the ProCAR system. Subcutaneous MDA-MB-468 tumors were established in NSG mice prior to I.T. injection with PBS, Pro⁻, or Pro^{PlGF} on day 40 post tumor engraftment. On day 44 Mice received an initial I.T. injection of 2.5×10⁶ untransduced (UT), or 2.5×10⁶ GFP28z ProCAR-T cells, followed by a second I.T. dose of UT or ProCAR-T cells 14 days later (day 58), as in FIG. 11F. Tumor growth was monitored by caliper measurements every 3-4 days, individual tumor trajectories are shown.

FIG. 14 shows that PlGF-based SAs bind to the surface of target cells. (A) GFP28z is fused to mScarlet at the C-terminal domain of the receptor for receptor visualization by fluorescence microscopy. (B) Confocal microscopy images of Jurkat cells stably engineered to express GFP28z-mScarlet fusions, CARs are shown in orange co-cultured with unlabeled MDA-MB-468 target cells. 100 ng/mL of purified PlGF SA protein was added to the corner of the indicated wells (+) and images were taken every 2-5 m, representative images from time 2 m and 30 m post PlGF addition are shown. Live cell images were acquired at the indicated time intervals following addition of ligand (PBS [vehicle] or purified PlGF) on a Nikon Ti Eclipse confocal microscope using 40× magnification. (C) Representative flow cytometric histograms of human T cells, HEK293T, HCT116, and MDA-MB-468 cells incubated with 100 ng/mL PlGF for 20 m and assessed for surface-bound GFP by flow cytometry, n=3. (D) Quantification of flow cytometric analysis, MFI mean fluorescence intensity. (D) Schematic of proposed heparan sulfate proteoglycan (HSPG) mechanism of action, in which PlGF-based SAs potentially bind to negatively charged HS strands on surface expressed HSPGs. Heparanase (HSPE), which cleaves heparan sulfate (HS) strands, disrupt this potential interaction. (F) Overnight killing assay of ffLuc⁺ MDA-MB-468 and (G) ffLuc⁺ Nalm6 target cells with the addition of recombinant human HSPE (20 ng/ml) at a 1 to 1 effector to target ratio. GFP28z CARs were supplied with 100 ng/mL PlGF. (H) Overnight killing assay of ffLuc⁺ MDA-MB-468 treated with sodium chlorate (NaClO₃). Error bars represent s.d. of n=3.

FIG. 15 shows the effect of TLR agonists on ProCAR-T cell viability and effector differentiation. (A) New EcN strains were generated with targeted gene knockouts against the msbB gene (LPS), the FliC gene (Flagellin), and both genes simultaneously to generate a double knockout (DKO) strain to reduce TLR4 and TLR5 stimulation on ProCAR-T cells. (B) Quantification of flow cytometric analysis of T cell viability following 24 hr incubation with heat-killed EcN strains and 500 ng/mL PlGF, shown relative to untreated (U/T) controls. T cells were stained with a viability dye for 10 min in PBS before analysis. (C) Differentiation phenotype of CAR⁺CD8⁺ T-cells exposed to stimulants as in (B). Pie charts representing the clockwise differentiation of CD8⁺ T cell populations from stem cell memory (T_{SCM}) CD62L⁺CD45RO⁻, to central memory (T_{cm}) CD62L⁺CD45RO⁺, to effector memory (T_{EM}) CD62L⁻CD45RO⁺, and to terminal effector (T_{eff}) CD62L⁻CD45RO⁻ cells. (D) Flow cytometric quantification (left) and representative histograms (right) of CD25 expression on GFP28z CAR-T cells in response to EcN strains. (E) Flow cytometric quantification of CD69 expression on GFP28z CAR-T cells in response to EcN strains alone (left) or in combination with 100 ng/mL PlGF (right). (F) Colorimetric quantification of TLR5 stimulation in response to decreasing CFU of EcN strains. TLR5-mediated NF-kB activation was quantified using HEK-Blue™ mTLR5 reporter cell (InvivoGen). Reporter cells were incubated with heat-killed EcN strains for 6 hr before secreted embryonic alkaline phosphate (SEAP) activity was analyzed according to manufacturer instruction. (G) Growth kinetics of EcN strains over 6 hr as measured by OD₆₀₀ on a Tecan plate reader. (H) Relative body weights of immunocompetent Balb/c mice following intravenous (IV) injection of 1×10⁷ CFU of EcN strains. Error bars represent s.e.m. of biological replicates, *p<0.05, **p<0.01, ***p<0.001 2-way ANOVA, with Holm-Sidak multiple comparison correction.

FIG. 16 shows the ProCAR platform safety and tolerance in immunocompromised mice. (A) Representative ex vivo IVIS images of bioluminescent bacteria detected in tumor, lungs, kidneys, spleen, and liver harvested from NSG mice bearing subcutaneous HCT116 CRC tumors at day 14 post intratumoral bacteria treatment. (B) Biodistribution of Pro^{x} found in tumor (Tu.), lungs (Lu.), kidneys (Ki), spleen (Sp), and liver (Li) samples calculated as colony-forming units (CFU) per gram of tumor. At day 14 (n=5) post bacteria treatment, tumor, liver, and spleen were homogenized and plated on LB agar plates containing the appropriate antibiotics for bacteria colony quantification. (C) Representative ex vivo IVIS images of GFP fluorescence detected in tumor, lungs, kidneys, spleen, and liver harvested from mice bearing subcutaneous HCT116 CRC tumors at day 14 post intratumoral bacteria treatment. (D) ELISA quantification of GFP concentration found in mouse tumor and matched healthy tissues. At day 14 post bacteria treatment, tumors, lungs, kidneys, spleens, and livers were homogenized from mice bearing subcutaneous HCT116 tumors and assessed for GFP concentration. Error bars represent s.e.m. of biological replicates.

FIG. 17 shows that severely immunocompromised mice tolerate systemically delivered probiotic E. coli Nissle 1917 (EcN). (A) Biodistribution of healthy organs harvested from NSG mice on day 7 post systemic delivery (tail vein injection) of 1×10⁶ or 5×10⁶ colony forming units (CFU) of EcN-SLIC (WT), demonstrating the lack of detectable bacteria in the liver and spleen. (B) Body weights of NSG mice bearing 4T1 subcutaneous tumors and treated with EcN strains as in (A). (C) IVIS images of mice treated as in (B) demonstrating tumor localization of bioluminescent WT EcN and the FliC KO strain on day 3 post intravenous (i.v.) delivery. (D) Quantification of the percentage of tumors colonized 24 or 48 hr post i.v. injection, demonstrating that 100% of tumors were successfully colonized within 48 hr (n=10 per treatment group).

FIG. 18 Optimization of synthetic antigen (SA) production. (A) A panel of constitutive promoters were screened for increased GFP production and growth kinetics of WT EcN-SLIC on a Tecan MicroPlate reader (488 nm). Fold change of GFP fluorescent signal is shown over the original expression plasmid, shown here as pTac 687 RBS*. (B) A single vector was constructed to optimize the production of two genes (PlGF-SA in combination with either CXCL9 or CXCL16^{K42A}) within a single EcN strain. GFP production was determined as in (A) and shown as fold change over the original expression plasmid, pTac 647 RBS*. (C) ELISA quantification of GFP concentration found in mouse tumors. At day 2 and day 7 post bacteria treatment, tumors were homogenized from mice bearing subcutaneous 4T1 tumors and assessed for GFP concentration by GFP-specific ELISA. Error bars represent s.e.m. of biological replicates, **p<0.01 1-way ANOVA, with Holm-Sidak multiple comparison correction.

FIG. 19 shows that probiotics release additional tumor microenvironment (TME) modulating therapeutics that directly recruit ProCAR-T cells to the tumor site. (A) Engineered probiotic EcN-SLIC (Pro^{x}) colonizes the solid tumor core and releases the human chemokine, CXCL16^{K42A}, designed to directly recruit CXRC6⁺ GFP28z ProCAR-T cells to the TME following systemic delivery. (B-E) MDA-MB-468 cells (5×10⁵) were implanted subcutaneously into the hind flank of NSG mice. When tumor volumes reached ˜60 mm³, mice were I.T. injected with 1×10⁵ CFU of Pro^{x} strains producing either PlGF (Pro^{PlGF}) alone, CXCL16^{K42A} (Pro^{CXCL16}) alone, a strain producing the PlGF+CXCL16^{K42A} combination (Pro^{Combo}), or a PBS control. On days 43 and 58 post Pro^{x} injection, 5×10⁶ GFP28z ProCAR-T cells were delivered by I.V. injection. Tumor growth was monitored by caliper measurements every 3-4 days (n>4/group). (C) Mean tumor trajectories, (D) survival curves, (E) fold change in mouse body weights are shown. Error bars represent s.e.m. of biological replicates, ****p<0.0001, 2-way ANOVA (B) with Holm-Sidak multiple comparison correction. Survival curve (C) *p<0.05, log-rank test.

FIG. 20 shows that probiotic-derived mutated human CXCL16 has bioactivity on activated T cells. (A) Detection by ELISA of human CXCL16 in culture supernatant of E. coli Nissle expressing hCXCL16 with or without SLC. (B) Detection of hCXCL16 by ELISA in homogenate of A20 tumors untreated (“none”) or treated with E. coli Nissle expressing hCXCL16 with (“+SLC”) or without SLC (“-SLC”). (C) Description of CXCL16 variants used. (D) Schematic of chemotaxis assay. (E) Cell migration of activated mouse T cells in response to lysate of indicated bacteria strain assessed by flow cytometry, shown as percentage of cell input. Media without any bacteria was used as a control. Wild-type E. coli Nissle lysate (WT) did not have therapeutic cargo. Bacteria were OD₆₀₀ matched prior to lysis. (A, E): Representative of 3 independent experiments. (A): 2-sided unpaired t-test: ****p<0.001. (B): 1-way ANOVA with Holm-Sidak post-hoc test. ***p<0.001. (E): 2-way ANOVA with Dunnett post-hoc test. *p<0.05.

FIG. 21 shows the activating mutation of human CXCL16 has bioactivity on activated human T cells. Cell migration of activated human T cells in response to lysate of indicated bacteria strain assessed by flow cytometry after 3 hour incubation, shown as percentage of cell input. Media without any bacteria was used as a control. Wild-type bacteria (WT) did not have therapeutic cargo. Representative of 2 independent human controls.

FIG. 22 shows that the activating form of hCXCL16 (hCXCL16^{K42A}) strain promotes tumor regression in subcutaneous B cell lymphoma model. (A) Treatment of subcutaneous syngeneic tumors. After tumors were palpable (˜100 mm³), mice were treated via intratumoral delivery every 3-4 days for 4 treatments. (B-C) Tumor growth of A20 tumors in BALB/c mice in response to (B) indicated hCXCL16 variants or (C) hCXCL16^{K42A} strain versus PBS or E. coli Nissle expressing SLC but no therapeutic (eSLC). Treatments, indicated by black arrows, began after tumors reached ˜100 mm³. (D-H) Flow cytometry analysis of tumor infiltrating lymphocytes in A20 model. Mice were treated with two intratumoral injections of indicated strain 4 days apart, with analysis day 8 post-initial treatment. (D) Ki-67⁺ percentage among CD4⁺ T_{con}(CD4⁺Foxp3⁻) and CD8⁺ T cells. (E) IFNγ expression among CD4⁺ T_{con}. following ex vivo restimulation. (F) Granzyme-B⁺ frequencies among CD8⁺ T cells (CD3⁺TCRß⁺CD8⁺). (G-H) IFNγ expression among CD8⁺ T cells following ex vivo restimulation with (G) PMA/ionomycin and (H) class-I restricted A20 idiotype peptide. (I) A20 tumors were implanted on both hind flanks, with intratumoral injections with indicated strain on a single flank. Growth of untreated A20 tumors is shown. (J) Growth of A20 tumors following treatment (black arrow) with indicated strain via a single intravenous injection. (B-C, I-J): Representative of 2-3 independent experiments. 3-6 tumors per group. p-values from a 2-way ANOVA with Holm-Sidak post-hoc test: *p<0.05, **p<0.01, ***p<0.001, ****p<0.0001. (D-H): Representative of 2 independent experiments, p-values from 1-way ANOVA with Holm-Sidak post-hoc test: *p<0.05, **p<0.01, ***p<0.001, ****p<0.0001.

FIG. 23 shows the activating mutation of human CXCL16 in syngeneic A20 tumors. (A-B) Individual tumor trajectories of A20 tumors in BALB/c mice in response to (A) indicated hCXCL16 variants or (B) hCXCL16^{K42A} strain versus PBS or E. coli Nissle expressing SLC but no therapeutic (eSLC). Treatments, indicated by black arrows, began after tumors reached ˜100 mm³. (C-D) Production of indicated cytokines among (C) CD4⁺ T_{con}. (CD4⁺Foxp3⁻) and (D) CD8⁺ T cells following ex vivo restimulation with PMA/ionomycin of tumor infiltrating lymphocytes in A20 tumors. (E) Production of indicated cytokines among CD8⁺ T cells following ex vivo restimulation with A20 idiotype peptide in A20 tumors. (F) Individual tumor trajectories of untreated A20 tumors in BALB/c mice. Tumors on opposing flank were treated with indicated strain or PBS. (G) Individual tumor trajectories of A20 tumors in BALB/c mice in response to indicated strain following single intravenous injection (indicated by black arrow).

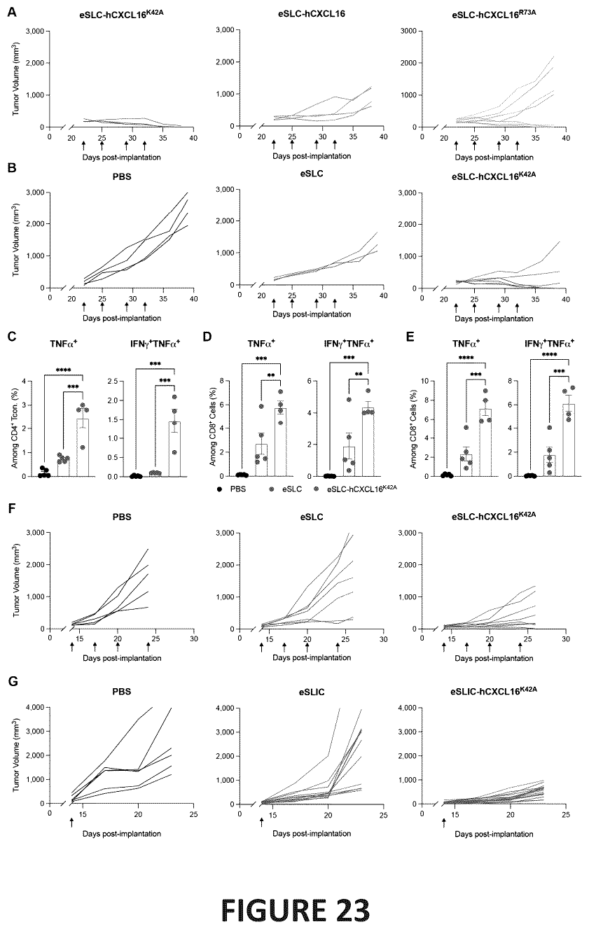

FIG. 24 shows that the activating form of hCXCL16 (hCXCL16^{K42A}) strain offers therapeutic benefit in colorectal and breast cancers. (A) Growth of subcutaneous MC38 tumors in C57BL/6 mice with indicated treatment starting after tumors were palpable. (B) Ki-67⁺ and (C) Granzyme-B⁺ frequencies among CD4⁺ conventional T cells (CD4⁺ T_{con}.; CD3⁺TCRß⁺CD4⁺Foxp3⁻) and CD8⁺ T cells (CD3⁺TCRß⁺CD8⁺) as assessed by flow cytometry in the MC38 model. Mice were treated with a single intratumoral injection of indicated strain, and tumors were analyzed 5 days later. (D) Granzyme-B⁺ frequencies among CD4⁺ T_{con} and CD8⁺ T cells 8 days post-initial treatment in the MC38 model. Mice were treated with two intratumoral injections of indicated strain 4 days apart. (E) Growth of subcutaneous EO771 tumors in C57BL/6 mice with indicated treatment. (A-E): Representative of 2 independent experiments. P-values from a 2-way ANOVA with Holm-Sidak post-hoc test: *p<0.05, **p<0.01, ***p<0.001, ****p<0.0001.

FIG. 25 shows individual tumor trajectories of syngeneic tumors treated with CXCL16 or controls. (A-B) Individual tumor trajectories of (A) MC38 or (B) EO771 tumors treated with indicated therapy via intratumoral injection. Treatments, indicated by black arrows, began after tumors reached ˜100 mm³. (C) Individual tumor trajectories of MC38 tumors in response to indicated strain following single intravenous injection (indicated by black arrow).

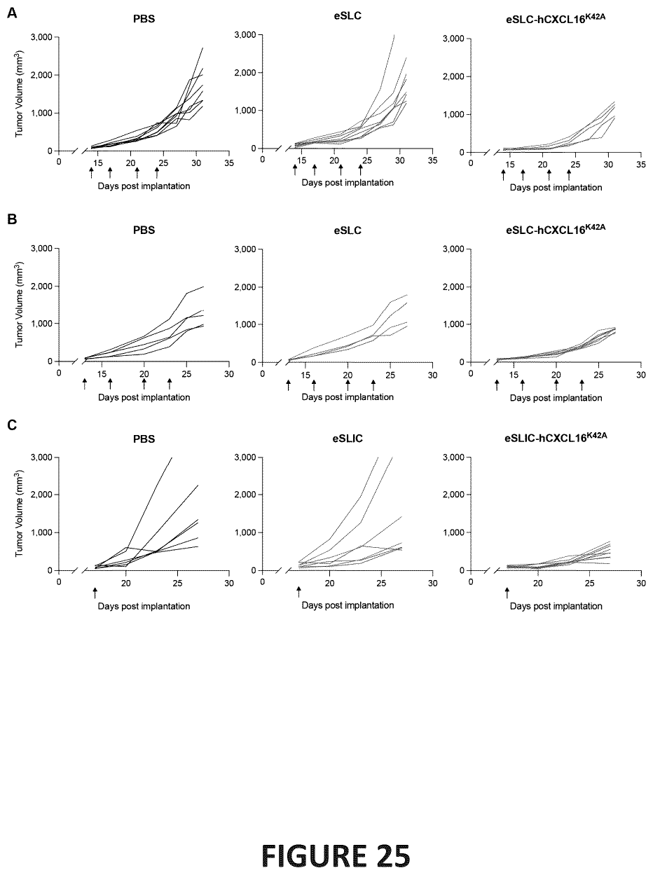

FIG. 26 shows that the combination of CXCL16 and CCL20 synergizes to promote anti-tumor immunity. (A) Growth of MC38 tumors in C57BL/6 mice with indicated treatment. eSLC-combo was a 1:1 mixture of the eSLC-hCXCL16^{K42A} and eSLC-CCL20 strains. Treatments began after tumors were palpable and are indicated by black arrows. (B) Frequency and (C) number of type 1 conventional dendritic cells at day 5 post-treatment initiation. Mice were treated with a single intratumoral injection of indicated strain. eSLC-combo as in (A). (D) Granzyme-B⁺ frequency among CD8⁺ T cells following treatment with indicated strain. (A): Representative of 3 experiments. P-values from a 2-way ANOVA with Holm-Sidak post-hoc test: *p<0.05, **p<0.01, ***p<0.001, ****p<0.0001.

FIG. 27 shows individual tumor trajectories of MC38 tumors in combination therapy approach. Individual tumor trajectories of MC38 tumors treated with indicated therapy via intratumoral injection. Treatments, indicated by black arrows, began after tumors reached ˜100 mm³. eSLC-combo is a 1:1 mixture of eSLC-hCXCL16^{K42A} and eSLC-CCL20.

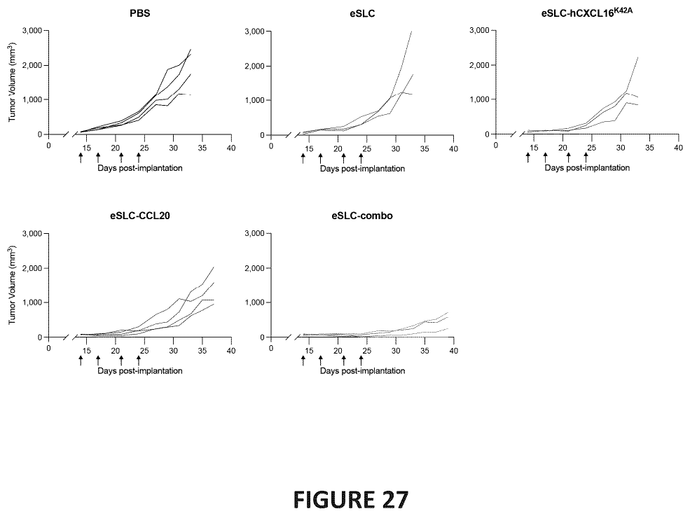

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 2.0 to 3.0 includes 2.0, 3.0, and all points between 2.0 and 3.0.

The term “about” as used herein when referring to a measurable value such as an amount, a temporal duration, and the like, is meant to encompass variations of .+−.20%, .+−.10%, .+−.5%, .+−.1%, or .+−.0.1% from the specified value, as such variations are appropriate to perform the disclosed methods.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of “consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of and “consisting essentially of” shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The inventions described herein relate to a platform or system for activating an immune response against tumor cells to thereby treat hyperproliferative disorders. The components of the system comprise programmable bacteria cells that produce one or more synthetic antigens and (optionally) one or more cytokines and immune T cells expressing a chimeric antigen receptor (CAR-T cell), wherein the CAR-T cells are engineered to recognize and respond to the antigen and activate an immune response against tumor cells as described hereinbelow.

Programmable Bacteria Cells

In some embodiments of the inventions described herein, synthetic antigens and/or cytokines are produced by one or more programmable bacterial cells. The programmable bacterial cells comprise heterologous nucleic acid sequences, which include one or more sequences that encode the synthetic antigen and/or cytokines and sequences that encode a synchronized lysis circuit (i.e., a quorum-sensing gene, a nucleic acid encoding a lysis gene, a promoter, and a terminator contained on a single operon). By including a synchronized lysis circuit, the programmable bacterial cells are capable of lysing in response to one or more internal or external stimuli, such as achieving a certain concentration or cell density in a tumor microenvironment, thereby releasing the synthetic antigens and/or cytokines and other cellular components into the surrounding environment (e.g., tumor microenvironment).

In some embodiments, the synthetic antigens produced by the programmable bacterial cells are green fluorescent proteins (GFP). In particular embodiments, the GFP are super-folder green fluorescent proteins (sfGFP). In one particular embodiment, the sfGFP is a dimeric version of sfGFP that comprises a 20-amino acid peptide-tag derived from the heparin binding domain of placenta growth factor-2 (sfGFP^{PlGF}). In another particular embodiment, the sfGFP is a soluble dimer comprising a cystine substitution at position D117 (sfGFP^{Dl}17C). In some embodiments, the synthetic antigen comprises an ALFA-tag linked to 20-amino acid peptide-tag derived from the heparin binding domain of placenta growth factor-2 (ALFA^{PlGF})

In some embodiments, the cytokine is IL-12. In some embodiments, the one or more cytokines include a chemokine. In some embodiments, the chemokine is a member of the CXC family of chemokines. In some embodiments, the chemokine is CXCL9. In other embodiments, chemokine is CXCL16. In some embodiments, the chemokine is an activating form of CXCL16, e.g., CXCL16^{K42A}. In some embodiments, the chemokine is CCL20.

The term “heterologous nucleic acid sequence” refers to a nucleic acid derived from a different organism that encodes for a protein and which has been recombinantly introduced into a cell, In some embodiments, the heterologous nucleic acid sequence is introduced by transformation in order to produce a recombinant bacterial cell. Methods for creating recombinant bacterial cells are well known to those of skill in the art. Such methods include, but are not limited to, different chemical, electrochemical and biological approaches, for example, heat shock transformation, electroporation, liposome-mediated transfection, DEAE-Dextran-mediated transfection, or calcium phosphate transfection. Multiple copies of the heterologous nucleic acid sequence (e.g., between 2 and 10,000 copies) may be introduced into the cell.

In some embodiments, the heterologous nucleic acid sequences are in a plasmid. In some embodiments, the heterologous nucleic acid sequences are in a single operon and are integrated into the genome of the programmable bacterial cells. In some embodiments, the programmable bacterial cells comprise at least one inducible promoter or non-constitutive promoter that is in operable linkage with one or more of the heterologous nucleic acid sequences.

In some embodiments, the programmable bacterial cells comprise one or more biosensor circuits that detect hypoxia, low pH and high lactate levels, which are characteristics of the tumor environment. The biosensor-containing bacterial cells will allow for more specific targeting to the tumor, the biocontainment of the bacterial cells in the tumor and minimize colonization outside the tumor. See, e.g., PCT Application Publication No. WO/2021/137937, hereby incorporated by reference in its entirety.

As used herein, the term “promoter” means at least a first nucleic acid sequence that regulates or mediates transcription of a second nucleic acid sequence. A promoter may comprise nucleic acid sequences near the start site of transcription that are required for proper function of the promoter. As an example, a TATA element for a promoter of polymerase II type. Promoters of the present invention can include distal enhancer or repressor elements that may lie in positions from about 1 to about 500 base pairs, from about 1 to about 1,000 base pairs, from 1 to about 5,000 base pairs, or from about 1 to about 10,000 base pairs or more from the initiation site.

The term “inducible promoter” refers to an operable linkage between a promoter and a nucleic acid sequence, whereby the promoter mediates the nucleic acid transcription in the presence or absence of at least one specific stimulus. In some embodiments, the inducible promoter mediates transcription of a nucleic acid sequence in the presence or absence of at least one, two, three, four, or five or more stimuli. In some embodiments, the one or more stimuli are produced in whole or in part by the programmable bacterial cells. In some embodiments, the only stimulus of the promoter is the presence of a certain concentration or density of programmable bacterial cell found in the subject of a patient (e.g., in a tumor).

An “operable linkage” refers to an operative connection between nucleic acid sequences, such as for example between a control sequence (e.g., a promoter) and another nucleic acid sequence that codes for a protein i.e., a coding sequence. If a promoter can regulate transcription of an exogenous nucleic acid sequence then it is in operable linkage with the gene.

In accordance with the purposes of the inventions described herein, the programmable bacterial cells are preferably non-pathogenic and colonize tumors. One of ordinary skill in the art would know how to attenuate pathogenic bacteria to create non-pathogenic bacteria. In some embodiments, the bacteria are attenuated by removing, knocking out, or mutating a virulence gene such as altering genetic components of the bacterial secretion system.

In some embodiments, the programmable bacterial cells belong to at least one genus selected from the group consisting of Salmonella, Escherichia, Firmicutes, Bacteroidetes, Lactobacillus, and Bifidobacteria. In some embodiments, the bacterial cells belong to more than one genus selected from the group consisting of Salmonella, Escherichia, Firmicutes, Bacteroidetes, Lactobacillus, and Bifidobacteria.

In some embodiments, the programmable bacterial cells belong to the genus Escherichia. In particular embodiments, the programmable bacterial cells are Escherichia coli Nissle (EcN) cells. In one embodiment, the EcN cells comprise a knockout of the FliC gene. In one embodiment, the EcN cells comprise a knockout of the msbB gene. In one embodiment, the EcN cells comprise a knockout of both the FliC gene and the msbB gene.

Some aspects of this invention implicitly relate to culturing the programmable bacterial cells described herein. In some embodiments, a culture comprises the programmable bacterial cells and a medium, for example, a liquid medium, which may also comprise: a carbon source, for example, a carbohydrate source, or an organic acid or salt thereof; a buffer establishing conditions of salinity, osmolarity, and pH, that are amenable to survival and growth; additives such as amino acids, albumin, growth factors, enzyme inhibitors (for example protease inhibitors), fatty acids, lipids, hormones (e.g., dexamethasone and gibberellic acid), trace elements, inorganic compounds (e.g., reducing agents, such as manganese), redox-regulators (e.g., antioxidants), stabilizing agents (e.g., dimethyl sulfoxide), polyethylene glycol, polyvinylpyrrolidone (PVP), gelatin, antibiotics (e.g., Brefeldin A), salts (e.g., NaCl), chelating agents (e.g., EDTA, EGTA), and enzymes (e.g., cellulase, dispase, hyaluronidase, or DNase). In some embodiments, the culture may comprise an agent that induces or inhibits transcription of one or more genes in operable linkage with an inducible promoter, for example doxicycline, tetracycline, tamoxifen, IPTG, hormones, or metal ions. While the specific culture conditions depend upon the particular programmable bacterial cells, general methods and culture conditions for the generation of microbial cultures are well known to those of skill in the art.

Chimeric Antigen Receptor-T Cells

Chimeric antigen receptors (CARs or CAR-Ts) are genetically engineered receptors. These engineered receptors can be readily inserted into and expressed by immune cells, including T cells in accordance with techniques known in the art. With a CAR, a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell.

In some embodiments, CAR-T cells are designed to target GFP antigens described herein. In some embodiments, the CAR-T cells are designed to target the sfGFP antigens described herein. In some embodiments, the CAR-T cells are designed to target the ALFA-tag antigens described herein.

CAR-T cells in accordance with the present invention may be derived from T cells obtained from a subject to be treated, or they may be derived from a different subject entirely. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan. In some embodiments, the cells collected by apheresis are washed to remove the plasma fraction, and placed in an appropriate buffer or media for subsequent processing. In some embodiments, the cells are washed with PBS. As will be appreciated, a washing step can be used, such as by using a semiautomated flow through centrifuge. In some embodiments, the washed cells are resuspended in one or more biocompatible buffers, or other saline solution with or without buffer. In some embodiments, the undesired components of the apheresis sample are removed.

In some embodiments, T cells are isolated from PBMCs by lysing the red blood cells and depleting the monocytes, e.g., by using centrifugation through a PERCOLL™ gradient. In some embodiments, a specific subpopulation of T cells, such as CD4⁺, CD8⁺, CD28⁺, CD45RA⁺, and CD45RO⁺ T cells is further isolated by positive or negative selection techniques known in the art. For example, enrichment of a T cell population by negative selection can be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected can be used. For example, to enrich for CD4+ cells by negative selection, a monoclonal antibody cocktail typically includes antibodies to CD8, CD11b, CD14, CD16, CD20, and HLA-DR. In some embodiments, flow cytometry and cell sorting are used to isolate cell populations of interest.

In some embodiments, PBMCs are used directly for genetic modification with the immune cells (such as CARs). In some embodiments, after isolating the PBMCs, T lymphocytes are further isolated, and both cytotoxic and helper T lymphocytes are sorted into naive, memory, and effector T cell subpopulations either before or after genetic modification and/or expansion.

In some embodiments, CD8⁺ cells are further sorted into naive, central memory, and effector cells by identifying cell surface antigens that are associated with each of these types of CD8⁺ cells. In some embodiments, the expression of phenotypic markers of central memory T cells includes CCR7, CD3, CD28, CD45RO, CD62L, and CD127 and are negative for granzyme B. In some embodiments, central memory T cells are CD8⁺, CD45RO⁺, and CD62L⁺ T cells. In some embodiments, effector T cells are negative for CCR7, CD28, CD62L, and CD127 and positive for granzyme B and perforin. In some embodiments, CD4⁺ T cells are further sorted into subpopulations. For example, CD4⁺ T helper cells can be sorted into naive, central memory, and effector cells by identifying cell populations that have cell surface antigens.

Methods of manufacturing T cells expressing chimeric antigen receptors are known in the art. In some embodiments, the immune cells, e.g., T cells, are genetically modified following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The DYNABEAD® system, a CD3/CD28 activator/stimulator system for physiological activation of human T cells. In other embodiments, the T cells are activated and stimulated to proliferate with feeder cells and appropriate antibodies and cytokines.

Therapeutic Methods and Compositions

The inventions described herein also encompass methods of treating a hyperproliferative disorder comprising administering to a subject the system of programmable bacterial and CAR-T cells described hereinabove. In some embodiments, the hyperproliferative disorder is selected from the group consisting of breast cancer, melanoma, renal cancer, prostate cancer, pancreatic adenocarcinoma, colorectal cancer, lung cancer, esophageal cancer, squamous cell carcinoma of the head and neck, liver cancer, ovarian cancer, cervical cancer, thyroid cancer, glioblastoma, and glioma.

The inventions described herein also encompass methods of reducing the rate of proliferation of a tumor cell comprising administering to a subject the system of programmable bacterial and CAR-T cells described hereinabove. In some embodiments, the tumor cells are from a hyperproliferative disorder consisting of breast cancer, melanoma, renal cancer, prostate cancer, pancreatic adenocarcinoma, colorectal cancer, lung cancer, esophageal cancer, squamous cell carcinoma of the head and neck, liver cancer, ovarian cancer, cervical cancer, thyroid cancer, glioblastoma, and glioma. In some embodiments, the tumor cells are from tumor cell lines generated from one of the foregoing hyperproliferative disorders.

The inventions described herein also encompass methods of killing a tumor cell comprising administering to a subject the system of programmable bacterial and CAR-T cells described herein. In some embodiments, the tumor cells are from a hyperproliferative disorder consisting of breast cancer, melanoma, renal cancer, prostate cancer, pancreatic adenocarcinoma, colorectal cancer, lung cancer, esophageal cancer, squamous cell carcinoma of the head and neck, liver cancer, ovarian cancer, cervical cancer, thyroid cancer, glioblastoma, and glioma. In some embodiments, the tumor cells are from tumor cell lines generated from one of the foregoing hyperproliferative disorders.

As used interchangeably herein, “treatment” or “treating” or “treat” refers to all processes wherein there may be a slowing, interrupting, arresting, controlling, stopping, alleviating, or ameliorating symptoms or complications, or reversing of the progression of proliferative disease, but does not necessarily indicate a total elimination of all disease or all symptoms. Non-limiting examples of treatment include reducing the rate of growth of a tumor or cancer cell or cell associated with a hyperproliferative disease, reducing the size of a tumor, or preventing the metastases of a tumor.

Programmable bacterial cells and CAR-T cells described herein are preferably administered in one or more therapeutically effective doses. As used herein the terms “therapeutically effective dose” means the number of cells per dose administered to a subject in need thereof that is sufficient to treat the hyperproliferative disorder. In some embodiments, a therapeutically effective dose can be at least about 1×10⁴ cells, at least about 1×10⁵ cells, at least about 1×10⁶ cells, at least about 1×10⁷ cells, at least about 1×10⁸ cells, at least about 1×10⁹ cells, or at least about 1×10¹⁰ cells.

In some embodiments, programmable bacterial cells and CAR-T cells may be delivered to a subject in the form of a pharmaceutical composition, which may comprise one or more pharmaceutically acceptable carriers, diluents, or excipients. Each component of the system described herein may be formulated separately. Alternatively, components of the system may be formulated for co-administration. Pharmaceutical compositions may be formulated as desired using art recognized techniques. Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents, and the like, are also available. Certain non-limiting exemplary carriers include saline, buffered saline, dextrose, water, glycerol, ethanol, and combinations thereof. Pharmaceutical compositions may be frozen and thawed prior to administration, or may be reconstituted in WFI with or without additional additives (e.g., albumin, dimethyl sulfoxide). Programmable bacterial cells and CAR-T cells are preferably formulated for parenteral (e.g., intratumoral or intravenous) administration, but other routes of administration known in the art may be utilized.

Particular dosage regimens, i.e., dose, timing, and repetition, will depend on the particular subject being treated and that subject's medical history. Empirical considerations such as pharmacokinetics will contribute to the determination of the dosage. Frequency of administration may be determined and adjusted over the course of therapy, and is based on reducing the number of tumor cells or tumor mass, maintaining the reduction of such tumor cells or tumor mass, reducing the proliferation of tumor cells or an increase in tumor mass, or delaying the development of metastasis.

A therapeutically effective dose may depend on the mass of the subject being treated, his or her physical condition, the extensiveness of the condition to be treated, and the age of the subject being treated. In general, CAR-T cells disclosed herein may be administered in an amount in the range of about 1×10⁶ cells/kg body weight to about 5×10⁸ cells/kg body weight per dose.

Articles of Manufacture

The inventions disclosed herein also encompass articles of manufacture useful for treating a hyperproliferative disorder comprising a container comprising programmable bacterial cells described herein, or a pharmaceutical composition comprising the same, as well as instructional materials for using the same to treat the hyperproliferative disorder in connection with administration with CAR-T cells described herein. In some embodiments, the articles of manufacture are part of a kit that comprises a bacterial culture vessel and/or bacterial cell growth media.

The inventions disclosed herein also encompass articles of manufacture useful for treating a hyperproliferative disorder comprising a container comprising CAR-T cells described herein, or a pharmaceutical composition comprising the same, as well as instructional materials for using the same to treat the hyperproliferative disorder in connection with the co-administration of programmable bacterial cells described herein.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

DNA Constructs

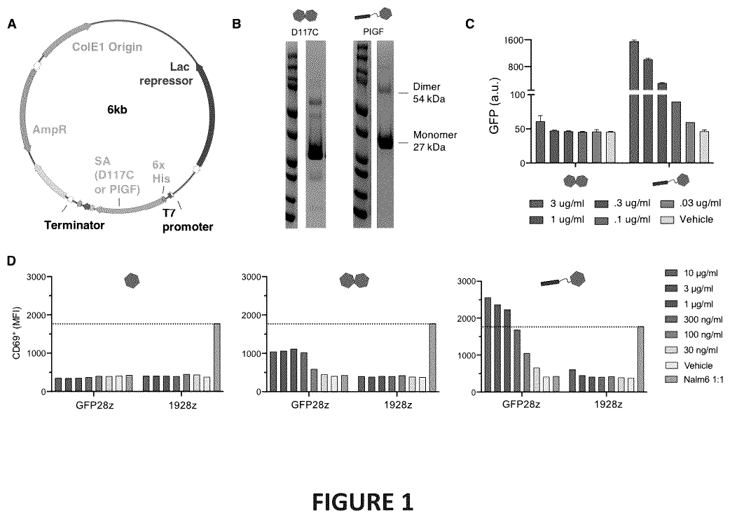

D117C was constructed using the sfGFP coding sequence with a cysteine substitution at position D117 (SEQ ID NO: 1) and PlGF was constructed by linking sfGFP to the PlGF₁₂₃₋₁₄₄ peptide sequence with a flexible glycine-serine linker at the C-terminus (SEQ ID NO: 2). The ALFA synthetic antigen (SEQ ID NO: 3) was constructed using the same design as the sfGFP-PlGF synthetic antigen. All bacterial payloads were cloned into an Axe/Txe stabilized p246-AT plasmid using Gibson assembly (NEB) methods in order to ensure high protein expression and facilitate protein. (FIGS. 1A & B).

The GFP specific ProCAR construct (SEQ ID NO: 4) was synthesized (IDT) based on a previously reported amino acid sequence for a GFP-binding nanobody, the ALFA CAR (SEQ ID NO: 5) was constructed using an ALFA-binding nanobody, the CD19 CAR was constructed from the FMC63 antibody, the GPC3 CAR was constructed from the GC33 antibody, and the ICAM1 CAR was constructed by using the ligand binding domain from LFA1 (LFA1₁₂₉₋₃₁₈). All antigen-recognition domains were fused to an IgG4 hinge and linker sequence and CD28 transmembrane and intracellular costimulatory domains in tandem with a CD3ζ signaling domain and were linked to mScarlet (SEQ ID NO: 6) by a T2A peptide sequence (SEQ ID NO: 7). CAR genes were cloned into a modified pHR_SFFV lentiviral transgene expression vector (Addgene plasmid #79121) with the EF1α promoter (SEQ ID NO: 8) inserted in place of SFFV using EcoRI and NotI restriction digest and InFusion cloning (Takara Bio).

### Example 2

Purification of Synthetic Antigens (SA)

D117C and PlGF SA variants were cloned into an inducible expression vector and transformed into eNiCo21(DE3) E. coli. Transformants were grown at 37° C. to an OD₆₀₀ of ˜0.9 and induced with 1 mM IPTG for 16 hr at 30° C. Cells were then centrifuged for 10 min at 4000 rpm and resuspended in lysis buffer (50 mM NaH₂PO₄, 300 mM NaCl, pH 8.0) for sonication. Lysates were spun for 30 min, following which the supernatant was loaded onto Ni-NTA (Qiagen) resin, washed in wash buffer (35 mM imidazole), and eluted in 250 mM imidazole for collection. The eluants were dialyzed in PBS using regenerated cellulose dialysis tubing (3500 Da MWCO) and then filtered through a 0.2 μm filter. 488 nm absorbance was used to determine the concentration, and diluted to a final of 1 mg/mL in PBS ready for use, aliquots were stored at −80° C.

### Example 3

Primary Human T Cell Isolation and Culture

Primary human T cells were isolated by negative selection for CD3+ populations (STEMCELL technologies, Easy Sep) from anonymous healthy human donor blood collected by leukopheresis and purchased from Stemcell Technologies. T cells were cryopreserved in CryoStor10 (Stemcell Technologies). After thawing, T cells were cultured in human T cell medium (hTcm) consisting of X-VIVO 15 (Lonza) and 5% Human AB serum (Gemini) supplemented with 50 units/mL IL-2 every 2 days for all experiments (Miltenyi Biotec).

### Example 4

Generation of Human CAR-T Cells

Pantropic VSV-G pseudotyped lentivirus was generated by transfection of HEK293T (ATCC, CRL-11268) with a pHR'SIN:CSW transfer vector and psPAX.2 and pMD2.G packaging plasmids using Lipofectamine 3000 (Invitrogen). 24 hr post transfection, T cells were thawed and activated with anti-CD3/CD28 DYNAL™ DYNABEADS™ (Gibco) at a 1:2 cell:bead ratio. At 48 hr post transfection and day 1 post T cell activation, viral supernatants were harvested and added to T cells at an MOI of 1.5-2 with 0.8 μg/mL of polybrene (MilliporeSigma). T cells were exposed to the virus for 24 hr before removal of polybrene and addition of fresh hTcm. DYNABEADS™ were removed from T cell cultures at day 6 post activation and T cells were transferred to GRex24 (Wilson Wolf), or GRex6, plates for expansion until day 13-14, at which point were considered rested and ready for use in assays or cryopreservation.

### Example 5

Pro^{x} Strain Generation and Administration

D117C, PlGF, and CXCL9 (SEQ ID NO: 9) expression vectors were transformed into electrocompetent EcN-SLIC (i.e., E. coli Nissle engineered with a synchronized lysis circuit) strains and cultured in LB media with 50 μg/ml kanamycin with 0.2% glucose, in a 37° C. shaking incubator. For therapeutic preparation, Pro^{x} strains were grown overnight in LB media containing appropriate antibiotics and 0.2% glucose. The overnight culture was sub-cultured at a 1:100 dilution in 50 mL of fresh media with antibiotics and glucose and grown to an OD₆₀₀ of ˜0.05, preventing bacteria from reaching quorum. Cells were centrifuged at 3000 rcf and washed 3 times with sterile ice-cold PBS. Pro^{x} strains were then diluted to a final concentration of 5×10⁶ CFU/mL in cold PBS, 40 μL of each strain was then injected intratumorally.

### Example 6

Cell Lines

Jurkat Clone E6-1 cells were purchased from ATCC (TIB-152) and lentivirally transduced to stably express either the GFP or CD19 CARs and cultured in RPMI-1640 (Gibco) supplemented with 10% fetal bovine serum (FBS, Gibco) and 1% penicillin/streptomycin (Gibco). Adherent target cell lines were purchased from ATCC unless otherwise stated and cultured in DMEM/F12 supplemented with 10% FBS and 1% penicillin/streptomycin: HEK293T cells (CRL-11268), MCF7 breast cancer cells (HTB-22), MDA-MB-468 triple negative breast cancer (HTB-132), and the HUH7 hepatoma cells were a gift from R. Schwabe (Columbia University). All target cell lines were transduced to stably express firefly luciferase (ffLuc), ffLuc⁺ Nalm6 acute lymphoblastic leukemia cells were a gift from M. Sadelain (Memorial Sloan Kettering Cancer Center) and cultured in RMPI-1640 supplemented as above.

### Example 7

Animal Models

All animal experiments were approved by the Institutional Animal Care and Use Committee (Columbia University, protocols AC-AAAN8002 and AC-AAAZ4470). Mice were blindly randomized into treatment groups. Animal experiments were performed on 6-8-week old female NOD.Cg-Prkdcscid Il2rgtm1Wjl/SzJ (NSG) mice (Jackson Laboratory) with subcutaneous hind-flank tumors from implanted human acute lymphoblastic leukemia cells (Nalm6), or Burkitts lymphoma cells (Raji, ATCC CCL-86), or triple negative breast cancer cells (MDA-MB-468). All tumor cells were prepared for implantation at a concentration of 5×10⁶ cells/mL in PBS and matrigel (Corning) at a 1:1 ratio. Tumors were grown to an average volume of approximately 100 mm³ before receiving an intratumoral injection of bacteria as described above. Two days later mice received an intratumoral injection of 2.5×10⁶ CAR⁺ T cells (5×10⁶ total T cells) in 50 μL of PBS. Tumor volume was calculated by measuring the length and width of each tumor using calipers, where V=length×width²×0.5. Tolerance to intertumoral injections of probiotics was assessed by monitoring mouse weight.

### Example 8

In Vivo Imaging and Biodistribution

All bacterial strains used were luminescent (integrated luxCDABE cassette) so they could be visualized with the In Vivo Imaging System (IVIS). To confirm bacterial localization, tumors, spleen and liver were weighed and homogenized using a GENTLEMACS™ tissue dissociator (Miltenyi Biotec; C-tubes). Homogenates were serially diluted, plated on LB agar plates and incubated overnight at 37° C. For plasmid retention analysis, tumor homogenates were also plated on LB-agar plates containing kanamycin. Colonies were counted and computed as CFU/g of tissue (limit of detection 10³ CFU/g).

### Example 9

T Cell Functional Assays

To assess cytotoxic responses to D117C and PlGF ffLuc+adherent cell lines (HEK293T, HUH7, MCF7, and MDA-MB-68) were allowed to adhere to collagen-coated plates (Thermo Scientific, NUNC™ F96 MICROWELL™ White Polystyrene) overnight. The next day media was removed and replaced with serum-free X-VIVO 15 containing D117C or PlGF SA variants and incubated at 37° C. while CAR-T cells and untransduced (UT) controls were prepared in serial half-log dilutions to establish a range of effector to target (E:T) ratios. T cells were added to each well and co-cultured for 16-20 hr before addition of BRIGHT-GLO™ (Promega) lysis buffer and luciferin substrate. Luminescence (RLU) was detected with a Tecan plate reader and specific lysis (%) was determined by normalizing RLU to co-cultures with UT T cells. Fold expansion in response to D117C and PlGF SAs was measured by counting T cells (Countess II, ThermoFisher Scientific) every 2-3 days for 14 days following a single stimulation with 0.1 μg/mL of both SAs on collagen coated plates. To assess in vitro cytokine production in response to SAs and/or EcN lysate, T cells were washed of IL-2-supplemented media and stimulated for 24 hr as described above. Cells were centrifuged for 5 min at 500 rcf and supernatants were transferred to v-bottom plates (Corning) to clear remaining cellular debris with a second 5 min spin at 500 rcf and transferred to a clean plate for storage at −80° C. until cytokine analysis on the LUMINEX™ 200 (MilliporeSigma, HCD8MAG-17K).

### Example 10

Flow Cytometry

All T cell assays were performed with rested T cells at day 13-15 post activation and purified monomeric GFP was added to all surface stains to stain GFP-CAR⁺ cells or Myc AF488 (Cell Signaling Technology clone 9B11) to stain CD19⁻, GPC3⁻, or ICAM1⁻CAR⁺ cells in flow cytometry-based assays. To assess CD69 expression in response to His-tag purified sfGFP variants, GFP28z CAR-T cells or Jurkat cells were plated on collagen-coated plates that were preincubated with a half-log dilution series of purified sfGFP variants. 16-20 hr later cells were surface-stained with anti-human CD3 BUV395 (BD clone SK7) and CD69 BV421 (Biolegend clone FN50). To assess activation marker status T cells were stimulated with 0.1 μg/mL of D117C or PlGF on collagen coated plates for 16-20 hr and surface-stained with anti-human CD3 BUV395 (BD clone SK7), CD4 BV785 (Biolegend clone RPA-T4), CD8 BV510 (BD clone RPA-T8), CD69 PE-Cy7 (Biolegend clone FN50), CD25 BV421 (Biolegend clone BC96), and CD107a APC (Biolegend clone H4A3). CD107a antibody was added to cell cultures 5 hr before study-end. To assess intracellular cytokine levels T cells were similarly stimulated in the presence of Brefeldin A (BD GolgiPlug) and surface-stained with CD3 BUV395 (BD clone SK7), CD4 BV785 (Biolegend clone RPA-T4) and CD8 BV510 (BD clone RPA-T8), before intracellular staining with TNFα BV421 (Biolegend clone MAb11), IL-2 PE-Cy7 (Biolegend clone MQ1-17H12), IFNγ APC (Biolegend clone B27). Intracellular staining was achieved using a BD fixation/permeabilization kit and following manufacturers instruction. To assess activation in response to EcN lysate T cells were similarly stimulated for 16-20 hr with 0.1 μg/mL of PlGF and with, or without, lysate produced by sonication and added at a final OD600 of 1. Cells were surface-stained using the activation marker panel above. Finally, to assess T cell phenotype in response to EcN lysate cells were similarly stimulated and surface stained with CD3 BUV395 (BD clone SK7), CD4 BV785 (Biolegend clone RPA-T4), CD8 BV510 (BD clone RPA-T8), CD45RO APC (Biolegend clone UCHL1), and CD62L BV605 (Biolegend clone DREG-56). All samples were acquired on a BD Fortessa.

### Example 11

Ex Vivo Tumor Processing and Immunophenotyping

To characterize the effects of live bacteria on human CAR-T cells, Nalm6 tumors were extracted on day 4 post bacteria treatment (day 2 post T cell treatment) and lymphocytes were isolated from tumor tissue by mechanical homogenization using a GENTLEMACS™ dissociator (Miltenyi Biotec) in complete hTcm. Cells were filtered through 70 μm cell strainers and washed in PBS before staining. Cells were stained with GHOST DYE™ Red 780 (Cell Signaling Technology) live/dead stain in PBS for 15 min on ice before washing and re-suspending in 2× human/mouse Fc block in stain buffer (BD) followed by surface-staining with monomeric GFP and anti-human CD45 AF700 (Biolegend clone HI30), CD19 PerCP-Cy5.5 (Biolegend clone SJ25C1), CD3 BUV395 (BD clone SK7), CD4 BV785 (Biolegend clone RPA-T4), CD8 BV510 (BD clone RPA-T8), CD45RO APC (Biolegend clone UCHL1), CD62L BV605 (Biolegend clone DREG-56), CD69 PE-Cy7 (Biolegend clone FN50), and CD25 BV421 (Biolegend clone BC96). To characterize T cell exhaustion on day 27 post treatment of MDA-MB-468, T cells were similarly isolated and stained with GHOST DYE™ and lineage/differentiation markers (without CD19) and with TIM-3 BV711 (Biolegend clone F38-2E2), LAG-3 BV421 (Biolegend clone 11C3C65), PD-1 PE-Cy7 (Biolegend clone EH12.2H7).

To assess in vivo production of cytokines in response to EcN, single cell homogenates were similarly achieved from Nalm6 tumors by mechanical homogenization in Milliplex lysis buffer (MilliporeSigma) and Halt™ protease inhibitor (ThermoFisher) and centrifuged at 500 rcf for 10 min to clear debris. Homogenates were transferred to clean tubes for storage at −80° C. until cytokine analysis on the LUMINEX™ 200 (MilliporeSigma, HSTCMAG28SPMX13). sfGFP levels were assessed from similarly prepared tumor homogenates and matched serum drawn from terminal cardiac-puncture using a GFP ELISA kit following manufacturers instruction (abeam).

### Example 12

Statistical Analysis

Statistical tests were calculated in GraphPad Prism 7.0. The details of the statistical tests are indicated in the respective figure legends. Where data was assumed to be normally distributed, values were compared using a one-way ANOVA for single variable or a two-way ANOVA for more than one variable with the appropriate post-test applied for multiple comparisons. For Kaplan-Meier survival experiments, we performed a log-rank (Mantel-Cox) test.

### Example 13

Synthetic Antigens (SA) and SA-Specific Chimeric Antigen Receptor (CAR) System Design

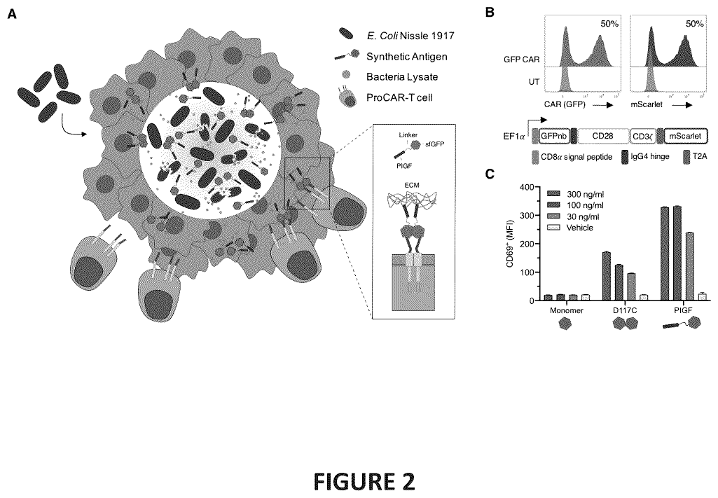

To ensure orthogonality to healthy human tissue, synthetic antigens (SA) were constructed by synthesizing two gene-blocks (IDT) encoding a tac promoter and E. coli-optimized genes for D117C and PlGF with N-terminal His-tags as described in Example 1. (FIG. 2). D117C and PlGF SA variants were cloned into an inducible expression vector, transformed, and purified as described in Example 2. Following purification of His-tagged SA, PlGF-linked sfGFP was shown to efficiently bind to collagen (FIG. 1C). To complete the GFP-based system, a SA-specific CAR was created using a sfGFP-binding nanobody sequence. Additional CAR were also designed and created as described in Example 1.

A lentiviral vector was constructed to co-express the CAR gene and a fluorescent mScarlet reporter from the EF1α promoter, separated by a self-cleaving T2A element as described in Example 1. By monitoring transduction efficiency of human T cells by mScarlet expression, surface expression and target specificity through CAR receptor binding to purified monomeric sfGFP was confirmed (FIG. 2B).

With the SA-system components in place, the extent of CAR-T cell activation elicited by each purified sfGFP variant was assessed. GFP CAR-T cells (GFP28z) were strongly activated by collagen-bound PlGF, moderately activated by dimeric D117C, and remained unchanged by exposure to monomeric sfGFP (FIGS. 2C and 1D).

### Example 14

GFP-Directed CAR-T Cells Mediate Killing of Target Cells in Response to Collagen-Bound sfGFP

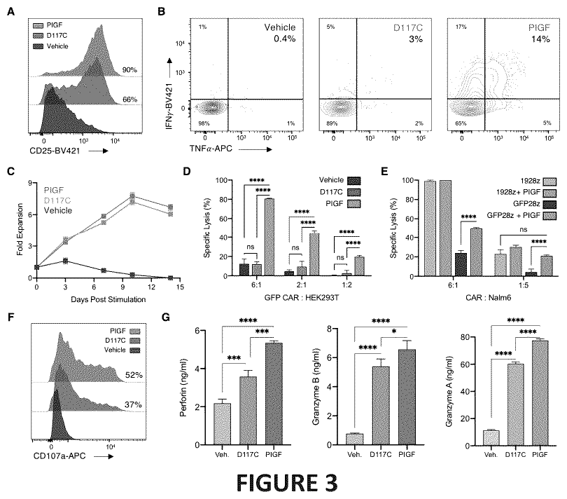

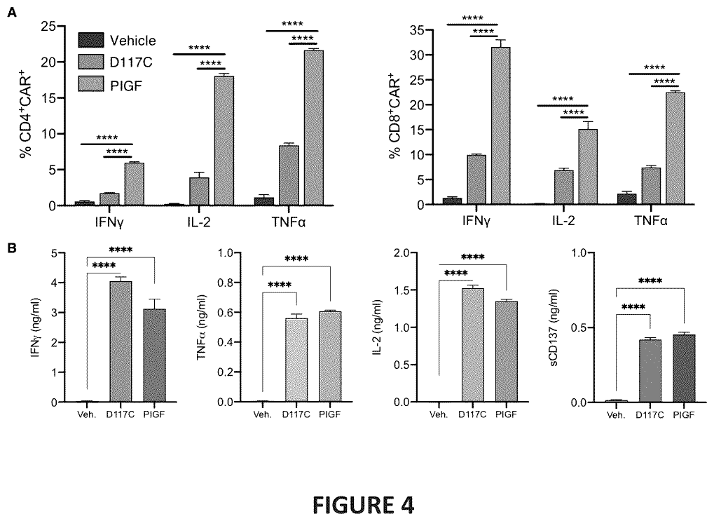

To further assess this pattern of activation, the surface expression of additional activation markers and intracellular cytokine production was interrogated by flow cytometry. The highest expression levels of CD25 were observed on GFP28z incubated with collagen-bound PlGF, with lower expression observed in response to soluble D117C (FIG. 3A). This trend was again mirrored in the intracellular levels of Th1 proinflammatory cytokines detected after 16 hr of coincubation, with GFP28z producing the highest levels of IFNγ, IL-2, and TNFα in response to collagen-bound PlGF (FIG. 4A). Similarly, these cells displayed an increased frequency of polyfunctional CD8+ T cells producing both IFNγ and TNFα (FIG. 3B). Stronger PlGF-mediated activation did not appear to drive higher rates of T cell expansion than exposure to D117C, as GFP28z cells expanded to a peak of 8-fold when stimulated with either SA (FIG. 3C). Without wishing to be bound by theory, this observation may be attributable to the comparable levels of IL-2 detected in cell culture supernatants by 24 hours of exposure to PlGF or D117C (FIG. 4B).

In prior studies, target cell lysis was not observed in response to soluble ligands, and minimal targeted lysis of ffLuc⁺ HEK293T cells was observed when GFP28z cells were supplied with PBS (vehicle) or D117C at any effector to target (E:T) ratio. In contrast, GFP28z cells incubated with the PlGF modification were able to drive target cell lysis in 80% of HEK293T cells at a high E:T ratio of 6:1, and 20% of cells at a lower E:T ratio of 1:2 (FIG. 3D). GFP28z also showed a dose-dependent response to PlGF with specific lysis of HEK293T cells observed at doses as low as 1.5 ng/ml (FIG. 4A).

The effect of PlGF on GFP28z against Nalm6 leukemia cells, a commonly used target of CD19-directed CARs, was subsequently examined. While PlGF had no effect on the potency of CD19 CAR-T cells (1928z), GFP28z achieved up to 50% lysis of the suspension cells when supplied with PlGF, with target cell death comparable to that of 1928z at the lower (1:5) E:T ratio (FIG. 3E). Specific lysis of HUH7 hepatoma cells, MCF7 breast cancer cells, and MDAMB-468 TNBC cells was also demonstrated, relative to levels of target cell death achieved by GPC3- and ICAM1-targeted CARs, respectively (FIG. 4B). These results demonstrate that PlGF provides a synthetic target that promotes tumor killing across different cell types.

Higher levels of surface expression of CD107a, a membrane-bound molecule commonly used as a proxy for cytotoxic degranulation, was detected on GFP28z treated with collagen-bound PlGF than cells exposed to D117C (FIG. 3F). Increased levels of perforin and granzymes A/B were also detected in the supernatant of GFP28z supplied with either SA, with the highest levels secreted in response to bound antigen (FIG. 3G). Taken together these results demonstrate that that PlGF provides a synthetic target that promotes tumor killing across different cell types and that CAR-T cells can mediate effective, tumor cell type-agnostic killing in response to synthetic targets such as PlGF.

### Example 15

PlGF-Based ProCAR System Mediates Localized Anti-Tumor Activity in a Subcutaneous Xenograft Model of Leukemia

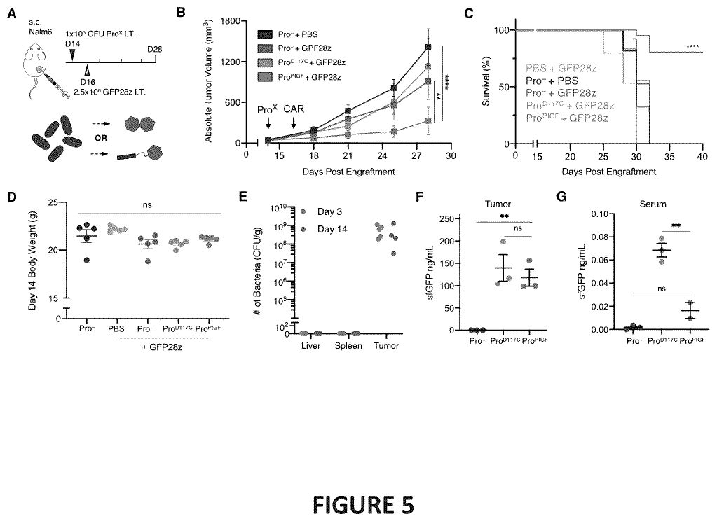

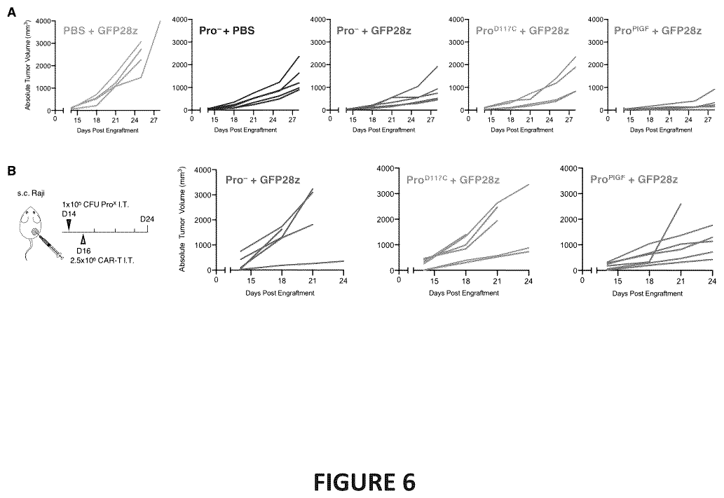

NSG mice bearing subcutaneous Nalm6 tumors were administered a single I.T. injection of 1×10⁵ CFU of Pro^{x} either producing the D117C (Pro^{D117C}) or PlGF (Pro^{PlGF}) SAs, or an empty control (Pro⁻) 48-72 hr before the mice received an intratumoral injection of 2.5×10⁶ GFP28z cells, or a PBS control (FIG. 5A). GFP28z in combination with Pro^{D117C} had no effect on tumor growth, with tumors growing at a similar rate to tumors receiving control Pro strains alone, or in combination with GFP28z (FIG. 5B, 6A). However, Pro^{PlGF} strains were able to mediate a potent antitumor response from GFP28z, leading to significantly slowed tumor growth of Nalm6 tumors and an increased survival benefit (FIG. 5B, 5C). This trend was also observed in mice bearing subcutaneous Raji tumors (FIG. 6B).

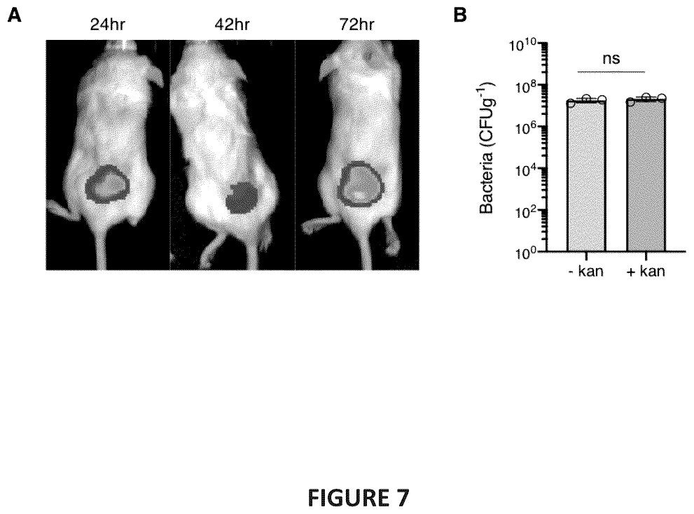

As a proxy for mouse health and system-tolerance, mouse body weight was monitored from the start of bacteria treatment and no significant weight loss was observed in mice treated with GFP28z alone, Pro alone, or any combination of the two cell therapies (FIG. 5D). Moreover, tumor-restricted growth of bioluminescent bacteria was observed in vivo (FIG. 7A) and bacteria were not detected outside of tumor homogenates on day 3 and 14 post treatment (FIG. 5E). Encouragingly, bacteria isolated from Pro^{PlGF}-treated tumors at day 14 additionally demonstrated SA-plasmid maintenance (FIG. 7B).

To assess the tumor-retention of probiotically-delivered SA variants, the level of detectable SA in tumor homogenates was quantified and serum samples from mice treated with Pro, Pro^{D117C} and Pro^{PlGF} strains were matched using GFP-specific ELISA. SA levels were as high or higher than those measured in in vitro assays in tumors treated with Pro^{D117C} and Pro^{PlGF} strains, and no difference in the intratumoral levels of either was observed (FIG. 5F). Without wishing to be bound by theory, it is believed that the difference in therapeutic efficacy observed between the two groups was likely not the result of differing SA abundance, and was instead attributable to the ability of PlGF to promote target cell killing compared to D117C. Higher concentrations of sfGFP were detected in the D117C serum of mice treated with Pro, suggesting that ECM-bound PlGF promotes tumor retention and reduces leakage into systemic circulation (FIG. 5G).

### Example 16

Engineered Strains of E. coli Nissle (EcN) 1917 Enhance CAR-T Cell Effector Function

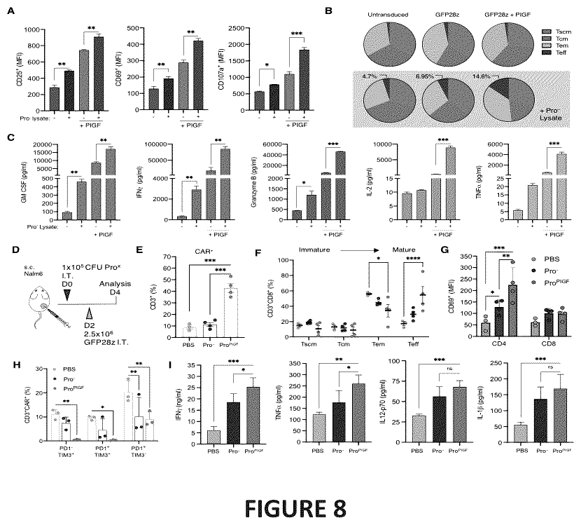

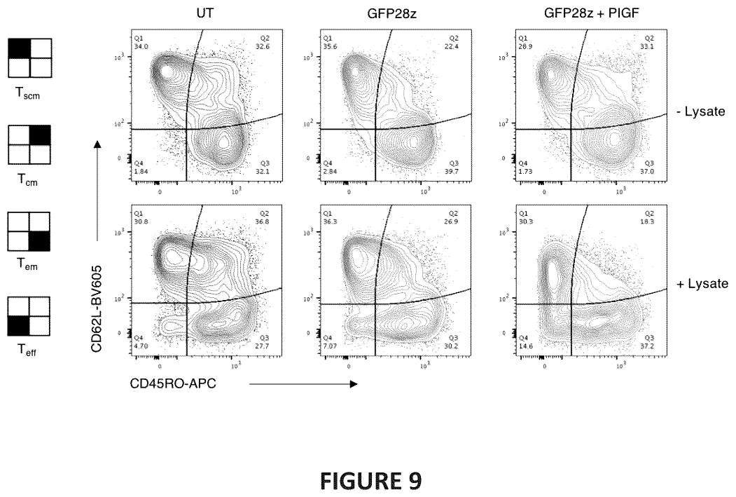

Activated T cells upregulate TLR4 and TLR5 expression of which LPS and flagellin from EcN are respective agonists. As such, intratumoral bacterial lysate may serve as an adjuvant to enhance ProCAR-T cell activity. To test this in vitro, the surface expression of CD69, CD25, and CD107a on GFP28z cells exposed to media alone, EcN lysate, PlGF alone, or the combination of PlGF and EcN lysate, was measured. GFP28z demonstrated significantly elevated levels of all three markers in response to EcN lysate alone, with the combination of lysate and collagen-bound PlGF stimulating the highest levels (FIG. 8A). Furthermore, CD8⁺ T cells exposed to EcN lysate displayed an effector-differentiated phenotype, with terminally differentiated effector populations (T_{eff}, CD45RO⁻CD62L⁻) expanding in both untransduced and GFP28z T cells (FIGS. 8B and 9). GFP28z exposed to the combination displayed the strongest enrichment of T_{eff} populations and furthest reduction in central memory populations (T_{cm}, CD45RO⁺CD62L⁻), while stem cell memory populations (T_{scm}. CD45RO⁻CD62L⁺) were maintained. The synergistic effect of PlGF in combination with EcN lysate was again mirrored in levels of Granzyme B and pro-inflammatory cytokines, GM-CSF, IFNγ, IL-2, and TNFα, detected in cell culture supernatants (FIG. 8C).

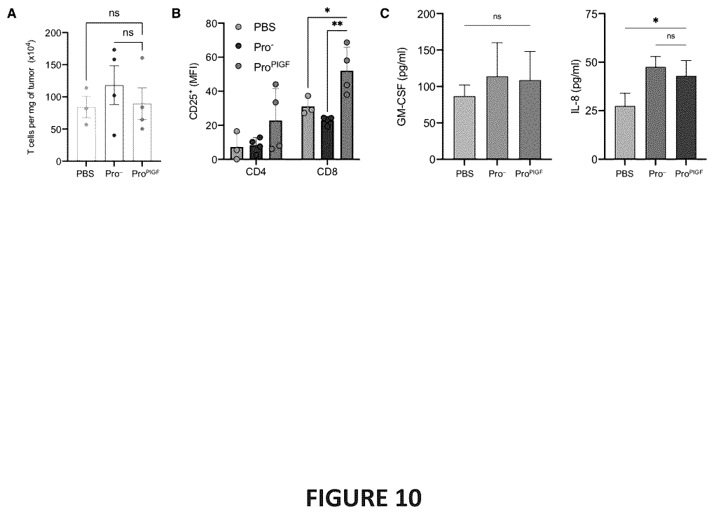

To study the effects of PlGF produced by live bacteria, the phenotype of GFP28z isolated from Nalm6 tumors treated with PBS, Pro⁻, or Pro^{PlGF} was interrogated. Tumors received an I.T. injection of Pro^{x} strains before receiving a second I.T. injection of GFP28z on day 2 post-treatment. Tumors were then homogenized and prepared for flow cytometry on day 4 (FIG. 8D). Tumors from all three groups were found to contain comparable levels of human CD45⁺CD3+ cells (FIG. 10A). However, a significant increase in the frequency of CD45⁺CD3⁺CAR⁺ cells was observed in tumors treated with Pro^{PlGF} suggesting CAR⁺ populations were specifically expanding in response to locally-released SA from tumor-colonizing bacteria (FIG. 8E). Moreover, CD8₊ GFP28z T cells from Pro^{PlGF}-treated tumors were significantly enriched for terminally differentiated T_{eff} populations, while cells from Pro⁻-treated tumors displayed a modest trend toward T_{eff} differentiation (FIG. 8F).

As a measure of activation, CD4⁺ GFP28z cells displayed significantly increased CD69 expression in response to Pro- and Pro^{PlGF} strains in vivo (FIG. 8G), though CD25 expression was found only to increase in response to Pro^{PlGF} (FIG. 10B). The exhaustion phenotype of GFP28z appeared inversely correlated with exposure to either of the Pro^{x} strains. The highest frequency of PD-1⁻TIM-3⁺, PD-1⁺TIM-3⁺, and PD-1⁺TIM-3⁻ cells was observed in the PBS control, while GFP28z from Pro^{PlGF}-treated tumors were found to be absent of the exhaustion marker TIM-3 entirely, and T cells from Pro⁻-treated tumors displayed an intermediate phenotype (FIG. 8H).

Cytokine profiling from treated tumors revealed a similar pattern. Tumors treated with either Pro^{x} strains were found to contain significantly increased levels of multiple human, pro-inflammatory cytokines, IFNγ, TNFα, IL12-p70, and IL-1β, relative to PBS groups, with the highest levels of IFNγ and TNFα detected in Pro^{PlGF}-treated tumors (FIG. 8I). Together, these observations highlight the dual functionality of the Pro^{PlGF} strain by providing both synthetic CAR targets and natural TLR stimulants that reshape the TME for enhanced CAR-T cell effector function.

### Example 17

The ProCAR System Produces a Durable Anti-Tumor Response in a Subcutaneous Xenograft Model of Triple Negative Breast Cancer

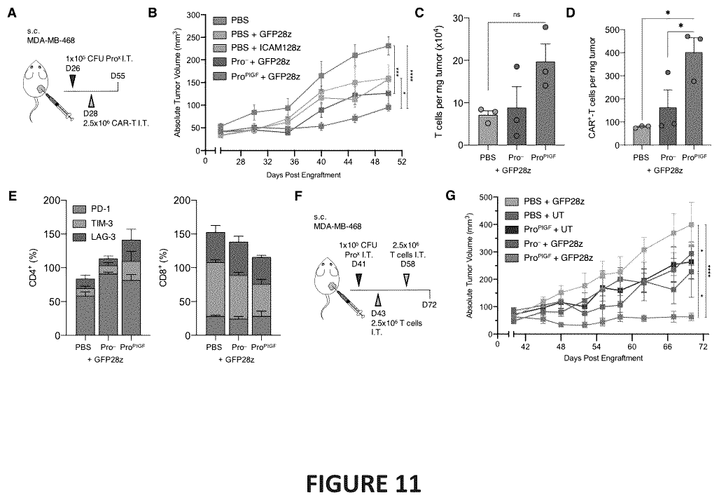

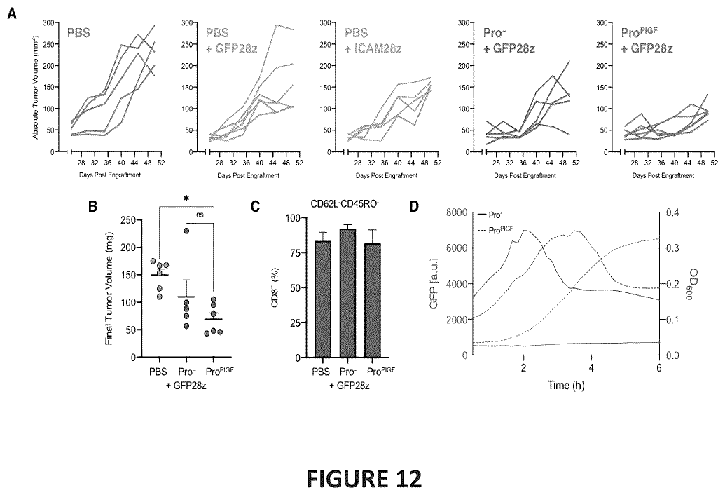

NSG mice bearing subcutaneous MDA-MB-468 TNBC tumors were administered a single I.T. injection of 1×10⁵ CFU of Pro^{x} bacteria either producing the D117C (Pro^{D117C}) or PlGF (Pro^{PlGF}) SAs, or an empty control (Pro⁻) 48 hours before the mice received an intratumoral injection of 2.5×10⁶ GFP28z cells, a PBS control, or an ICAM1-directed CAR (ICAM28z, FIG. 11A). The combination of Pro^{PlGF} and GFP28z demonstrated enhanced antitumor efficacy relative to ICAM28z, despite high ICAM1 expression on MDA-MB-468 cells. While Pro^{PlGF} again mediated the strongest antitumor activity of GFP28z, the therapeutic effects of TLR stimulation alone were evident in the combination treatment of GFP28z with Pro⁻ (FIGS. 11B and 12A). However, by day 51 post engraftment (day 25 post treatment) the therapeutic benefit of Pro^{PlGF} and GFP28z appeared reduced, and by day 55 only a small difference was observed between the weights of excised tumors from groups treated with GFP28z in combination with PBS, Pro⁻, or Pro^{PlGF} (FIG. 12B).

Upon phenotypic interrogation of the excised tumors, higher counts of human CD45⁺CD3⁺ cells (FIG. 11C) were observed and significantly increased counts of CAR⁺ cells in Pro^{PlGF}-treated tumors (FIG. 11D). As anticipated, the majority of these cells were classed as CD62L⁻CD45RO⁻ T_{effs} across all three groups (FIG. 12C), and displayed high levels of the exhaustion markers LAG-3, TIM-3, and PD-1 (FIG. 11E), suggestive of the T cell dysfunction commonly observed in the immunosuppressive TME. Encouragingly, ex vivo assessment of Pro⁻ and Pro^{PlGF} bacterial isolates from the same tumors revealed preserved functionality of quorum-based lysis and PlGF production (FIG. 12D).

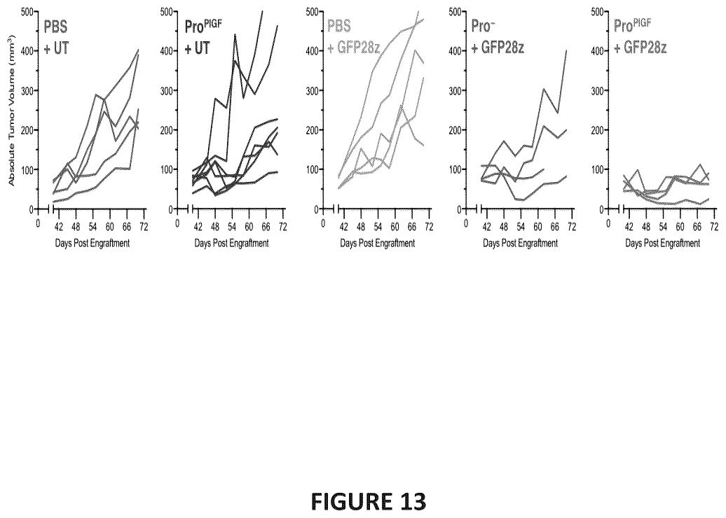

In order to prolong the antitumor activity of the ProCAR system, the dosage regimen was subsequently altered. While a single dose of Pro^{x} strains was administered to mice, the frequency of T cell treatment was increased to two doses spaced two weeks apart (FIG. 11F). With this, the Pro^{PlGF} and GFP28z combination was able to achieve a durable antitumor response, with no tumor growth observed 70 days post engraftment (FIGS. 11G and 13).

### Example 18

Characterization of SA Mechanism

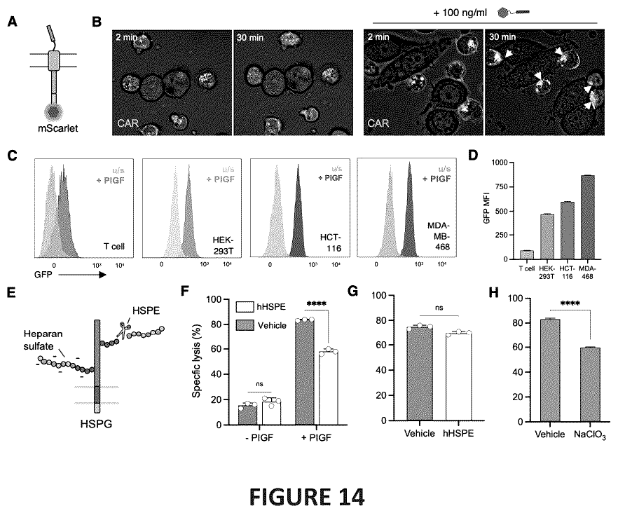

To visualize the interaction of GFP28z with MDA-MB-468 target cells with and without the SA, a GFP CAR receptor was fused to mScarlet at the C-terminus to track CAR-receptor subcellular localization by confocal microscopy (FIGS. 14 A & B). Thirty minutes post addition of purified PlGF, GFP28z cells appeared to directly interact with target cells, while untreated control GFP28z cells remain unchanged. The observed CAR-receptor clusters at the junctions between GFP28z and TNBC target cells suggests that PlGF coats the surface of MDA-MB-468 cells and causes polarization of CAR receptors akin to classical synapse formation between T cell and target.

Moreover, PlGF strongly coats the surface of MDA-MB-468 TNBC and HCT116 CRC cells, moderately binds to HEK293T cells, and only weakly binds the surface of untransduced, human T cells by flow cytometry (FIGS. 14 C & D). Without wishing to be bound by theory, this may explain why bystander killing of T cells in in vitro assays in response to an ECM-binding PlGF is not observed.

The role of heparan sulfate (HS) and heparan sulfate proteoglycans (HSPGs) found overexpressed on the majority of carcinoma cells was also examined. As growth factors, including placental growth factor (PlGF), interact with HSPGs on the cell surface through positively-charged heparin binding domains, this interaction may provide an additional mode of action for direct-tumor targeting (FIG. 14F). Accordingly, the effect of human heparanase (hHSPE) on GFP28z-driven target cell lysis was assessed. In an overnight killing assay of ffLuc⁺ MDA-MB-468 cells, significantly reduced target cell lysis following enzymatic cleavage of HSPGs was observed (FIG. 14E). Addition of hHSPE did not affect 1928z-driven lysis of CD19⁺ Nalm6 cells (FIG. 14F)—suggesting the reduced response of GFP28z to PlGF in the presence of hHSPE is likely due to the weakened interaction of PlGF with the enzymatically-modified target cells. In order to further examine the SA mechanism, sodium chlorate (NaClO₃) was used to remove negatively charged sulfate groups, thus blocking charge-based interactions with PlGF (FIG. 14H). In response to that treatment, a 20-25% reduction in target cell lysis was observed with the addition of NaClO₃ relative to GFP28z+PlGF alone, suggesting that HS and HSPGs may directly contribute to PlGF binding to the cell surface, in addition to potential contributions from surface bound collagens and ECM proteins.

### Example 19

Optimization of E. coli Nissle 1917 (Pro⁻) Strains

The immune system is finely regulated with a series of natural logic-gates to carefully balance an effective immune response against prevention of autoimmunity, in which multiple stimulatory signals are required to avoid T cell death or anergy, while over-stimulation also quickly leads to the phenomenon of activation-induced cell death (AICD). As CAR-T cells are particularly vulnerable to Fas/FasL-mediated AICD due to the streamlined antigen receptor and co-stimulatory domains, bacterial adjuvants, including potent TLR4 and TLR5 agonists provided by EcN (LPS and flagella, respectively), may lead to increased frequency of AICD events through additional MyD88 and NFκB signaling that further increases Fas/FasL expression in T cells.

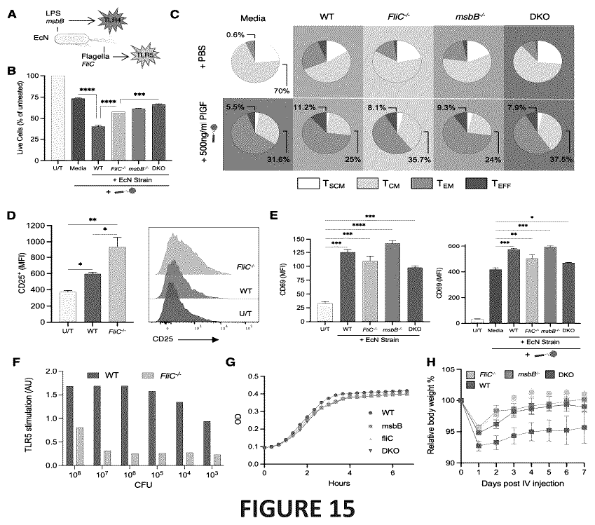

In order to address concerns of AICD, variant EcN strains were generated by targeted gene knock-out of the FliC gene (Flagellin), the msbB gene (LPS), or both genes simultaneously in a double knockout (DKO) strain (FIG. 15A). The flagellar filament structural protein encoding gene fliC and the lipid A biosynthesis myristoyltransferase encoding gene msbB were deleted using the λ-Red recombination system. Linear DNA containing chloramphenicol resistance gene was PCR amplified using pKD3 plasmid as a template and electroporated into bacteria that harbors pKD46 plasmid. Bacteria were recovered and plated in LB agar containing 12.5 μg/mL of chloramphenicol (Cm) and incubated overnight at 37° C. Chromosomal deletion of the genes was verified by colony PCR. To construct the fliC and msbB double-knock out strain, Cm resistance gene was removed from ΔmsbB strain by Flp-FRT recombination using pCP20 plasmid, and fliC gene was knocked out subsequently with the same method described above. The growth rate for the constructed strains was measured with Tecan MicroPlate reader starting from initial OD of 0.1 in LB without antibiotics.

The CRIM plasmid system was utilized to integrate synchronized lysis circuit (SLC) into the genome of Nissle ΔfliC. The SLC was integrated at φ80 site using pAH162 plasmid and the integration was verified by colony PCR. To characterize the lysis behavior of Nissle ΔfliC SLIC, OD₆₀₀ was measured every 20 mins by Tecan MicroPlate reader.

Using heat-killed bacteria, the effects of each strain on the viability of GFP28z T cells incubated with and without 500 ng/mL of PlGF-SA was assessed after 24 hr (FIG. 15B). PlGF alone (media control) significantly decreased the viability of GFP28z cells to ˜72% of untreated controls (U/T), while the viability of untransduced cells was unaffected, suggesting that high concentrations of PlGF causes AICD of GFP28z cells. This effect was amplified 2-fold with the addition of the wild type (WT) EcN strain, suggesting the combination of PlGF and WT-EcN leads to AICD in ˜60% of ProCAR-T cells. However, the reduction on T cell viability was significantly recovered with the substitution of WT for FliC^{-/-}, msbB^{-/-}, and DKO strains—with the KO of both TLR agonists providing the greatest reduction in cell death, in which ˜67% of T cells survived the combination of PlGF with DKO EcN.

ProCAR-T cell phenotype 24 hr post incubation with the modified EcN strains, with or without additional PlGF stimulation, was also examined (FIG. 15C). WT EcN in combination with PlGF caused a significant expansion of terminally differentiated effector populations (T_{EFF}), and a significant reduction in central memory populations (T_{CM}). ProCAR-T cells incubated with FliC^{-/-} and DKO strains in combination with PlGF displayed a similar differentiation profile to T cells incubated with PlGF alone, whereas T cells incubated with the msbB^{-/-} strain displayed a similar phenotype to cells incubated with the WT strain. Taken together, reducing TLR5 stimulation through FliC knock-out may be a mechanism to reduce potential AICD and prolong ProCAR-T cell activity in vivo.

Knock-out (KO) strains preserve T cell activation by monitoring the induction of CD25 expression in response to WT and FliC^{-/-} strains (FIG. 15D), and the induction of CD69 in response to WT, FliC^{-/-}, msbB^{-/-}, and DKO strains. The KO strains are additionally able maintain the adjuvant effects previously observed with WT EcN in combination with the PlGF-modified SA (FIG. 15E) and are likely to lead to increased therapeutic responses of ProCAR-T cells in vivo.

To confirm reduced TLR5 stimulation by the FliC^{-/-} strain, heat-killed WT and FliC^{-/-} EcN were incubated with HEK-BLUE™ mTLR5 reporter cells to monitor downstream NF-kB activity. Reporter cells exposed to the even the highest numbers of the FliC^{-/-} strain demonstrated reduced TLR5 stimulation relative to the WT EcN control (FIG. 15F). Characterization of the growth kinetics of the KO strains demonstrated no significant difference in growth rate of engineered strains relative to the WT (FIG. 15G). In addition to assessing the effects of these KO strains on T cell phenotype, the effects on mouse health were examined by monitoring mouse body weight following intravenous delivery of high doses of bacteria (1×10⁷ CFU). The FliC^{-/-}, msbB^{-/-}, and DKO strains demonstrated greater recovery of mouse body weight over the WT EcN control (FIG. 15H) in immunocompetent Balb/c mice.

### Example 20

Characterization of WT EcN and PlGF-Modified Synthetic Antigen Tumor Localization in NSG Mice

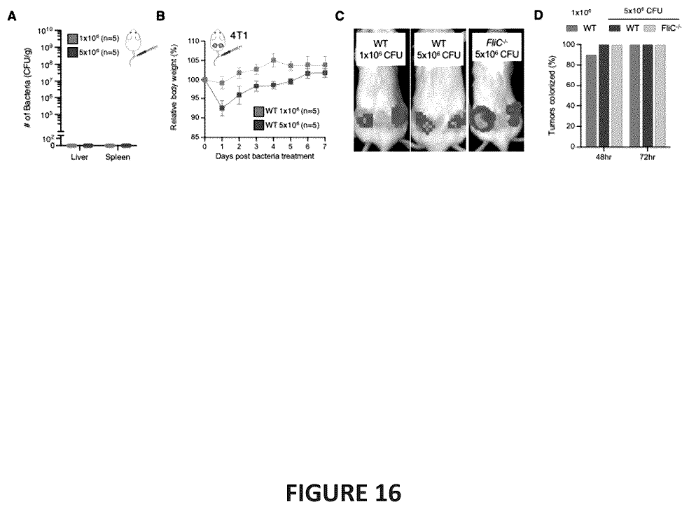

To further investigate the tumor-retention of intratumorally delivered Pro^{x} strains (WT EcN), the biodistribution of bacteria growing in the tumors, lungs, kidneys, spleens, and livers of mice were monitored. Bacteria were not detected in healthy organs with ex vivo imaging on day 14 post-treatment of mice bearing subcutaneous HCT116 CRC tumors (FIG. 16A). This observation was further confirmed by plating tumor and healthy tissue homogenates on antibiotic plates for colony counting (FIG. 16B).

In addition to bacteria biodistribution, the tumor retention of soluble and PlGF-modified SAs was examined in order to confirm minimal off-tumor activation of ProCAR-T cells. No GFP fluorescence was detected in the healthy organs or tumors of mice treated with Pro⁻, while high GFP signal was detected only in the tumors of mice treated with Pro^{PlGF} (FIG. 16C). This observation was further confirmed by quantifying GFP concentration in tumor and healthy tissue homogenates through GFP-specific ELISA (FIG. 16D).

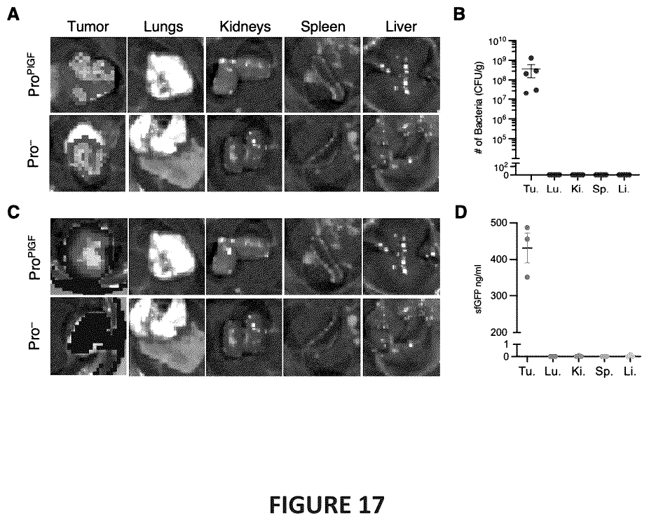

In addition, no dose-limiting toxicity of systemically-delivered EcN was observed 7 days post-injection (FIGS. 17A & B). Moreover, tumor colonization and retention of bioluminescent bacteria in NSG mice bearing subcutaneous 4T1 TNBC tumors was also observed (FIGS. 17 C & D). This is the first demonstration of bacteria colonization in a severely immunocompromised animal model.

### Example 21

Optimization of SA Expression and Generation of a Single Vector for the Expression of Multiple Therapeutics

To achieve higher expression of the PlGF synthetic antigen in the ProCAR system, the promotor and ribosome binding site (RBS) were optimized to achieve higher rates of mRNA transcription and protein translation, respectively. A plasmid that expresses two genes under two separate promoters was also created in order to generate a strain of bacteria that produces a combination of therapeutic payloads. A set of constitutive promoters was also screened in order to assess protein production against potential growth burden.

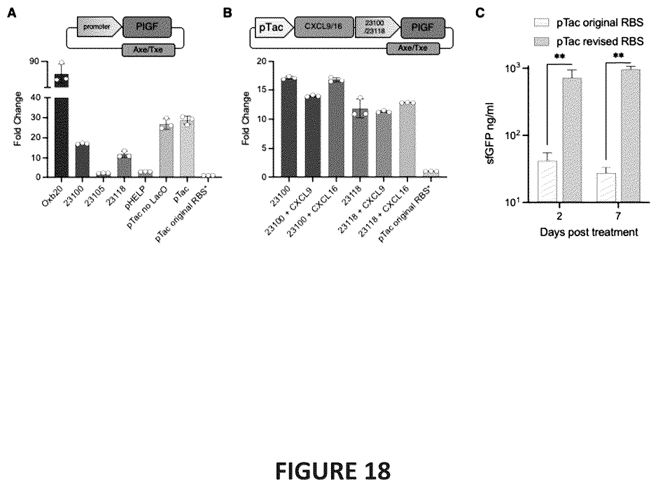

After monitoring GFP expression and bacterial growth, it was determined that some promotors provided optimal GFP expression and growth kinetics; 23100 & 23118 produced ˜15-fold higher GFP production, and the original pTac promoter with the optimized RBS demonstrating 30-fold higher GFP expression (FIG. 18A). Based on these observations, various vectors were created in order to produce a human chemokine (CXCL9 or CXCL16, SEQ ID NO: 10) under the control of the original pTac promoter and RBS, in combination with the PlGF SA under the control of optimized 23100 or 23118 promoters. The various vectors were screened for GFP production (FIG. 18B).

To assess the optimized strains in vivo, subcutaneous TNBS tumors were treated with 1×10⁵ CFU of WT EcN-SLIC strains equipped with the original SA expression plasmid (pTac original RBS). SA production was compared to an optimized expression plasmid (pTac revised RBS) following GFP-specific ELISA on tumor homogenates harvested on day 2 and day 7 post bacteria injection. The optimized Pro^{PlGF} strain produced significantly higher intratumoral levels of SA relative to the original Pro^{PlGF} strain with sustained production over 7 days (FIG. 18C).

### Example 22

Multiplexing the Procar Platform for Combinatorial Production of Synthetic Antigen (SA) and an Activating Form of Human Chemokine (CXCL16^{K42A})

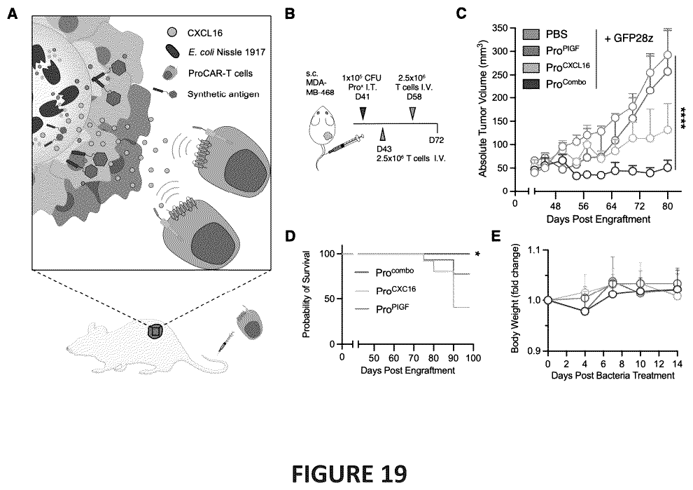

To achieve enhanced T cell trafficking to the tumor site and improve therapeutic activity of systemically delivered ProCAR-T cells, a combination strain (Pro^{combo}) was created in order to release the SA in combination with an activating form of human CXCL16 (CXCL16^{K42A}, SEQ ID NO: 11). CAR-T cells inherently express high levels of the CXCR6 chemokine receptor, and will traffic in response to concentration gradients leading to the site of intratumoral release by tumor-colonizing probiotics (FIG. 19A). Following an intratumoral injection of Pro^{x} strains in mice bearing subcutaneous MDA-MB-468 TNBC cells, ProCAR-T cells were delivered systemically by tail vein injection in two doses spaced two weeks apart and tumors were monitored for growth by caliper measurements every 3-4 days (FIG. 19B). Systemically delivered ProCAR-T cells were able to sustain a robust antitumor response in combination with the Pro^{Combo} strain producing both CXCL16^{K42A} and high levels of the PlGF-SA, relative to combinations with PBS, Pro^{PlGF} and Pro^{CXCL16-K42A} controls (FIG. 19C). Treatment with Pro^{Combo} strain in combination with ProCAR-T cells was able to achieve a survival benefit over combination treatment with the control Pro strains (FIG. 19D). Moreover, weight loss was not observed across any treatment group, suggesting that GFP28z ProCAR-T cells does not lead to off-target toxicities when delivered systemically (FIG. 19E).

### Example 23

Bacterial Strains

In order to further examine the utility of CXCL16 and variants thereof, additional experiments were carried out. Murine (Asn27-Pro114, UniProt Accession Number Q8BSU2) and human (Asn30-Pro118, UniProt Accession Number Q9H2A7) CXCL16, and murine CCL20 (Ala27-Met96, UniProt Accession Number O89093) were cloned into plasmid p246 via Gibson Assembly with the constitutively active pTac promoter. Following sequence confirmation of correct insertion, electrically competent E. coli Nissle 1917 were transformed with the p246 plasmid. The SLC plasmid (p15a) has been previously described. Strains with only a therapeutic plasmid (p246) were grown in LB broth with kanamycin (50 μg/mL). Strains with the therapeutic and SLC plasmids were grown in LB broth with kanamycin (50 μg/mL) and spectinomycin (100 g/mL) with 0.2% glucose. Mutant human CXCL16^{K42A} and CXCL16^{R73A} plasmids were generated from the wild-type hCXCL16 p246 plasmid by the New England BioLabs Q5 Site-Directed Mutagenesis Kit, as per the manufacturer's instructions.

### Example 24

Chemotaxis Assay

T cells were isolated from wild-type adult C57BL/6 mouse spleen and lymph nodes using the DYNABEADS® FlowComp Mouse Pan T (CD90.2) Kit as per the manufacturer's protocol. Isolated T cells were cultured with anti-CD3/CD28 beads (DYNABEADS® Cat. #11452D) in a 1:1 ratio in 10% complete RPMI (RPMI 1640 medium supplemented with 10% FBS, Pen/Strep, non-essential amino acids, Glutamax, HEPES, Sodium Pyruvate and 2-Mercaptoethanol). After 5 days, the beads were removed and T cells were re-plated at 10⁶ cells/mL for 4 days in 10% complete RPMI supplemented with 100 IU/mL of rhIL-2. T cells were then washed and resuspended at 5.9×10⁶ cells/mL in serum-free complete RPMI in preparation for the chemotaxis assay. Human T cells from STEMCELL Technologies were prepared identically using corresponding reagents for human cells.

Overnight cultures of each bacterial strain (without SLC) were grown in LB with appropriate antibiotics and then sub-cultured using a 1:100 dilution for 90 minutes. Bacteria were washed twice in serum-free complete RPMI, OD₆₀₀ matched, and lysed via sonication in serum-free complete RPMI. The lysate centrifuged to remove debris (20,817×g for 10′ at 4° C.) and 235 L of the supernatant was entered into the lower chamber of the trans-well plate (Corning HTS Trans-well 96 well area=0.143 cm²; Pore Size=5 μm). T cells (75 μL of above preparation) were added to the upper chamber and the plate was placed for 3 hours in a humidified 37° C. 5% CO₂ incubator. The bottom chamber was then harvested, washed and stained with anti-mouse CD3 violetFluor450 (Tonbo clone 17A2), CD4 APC (Tonbo clone RM4-5) and CD8 PE (Tonbo clone 53-6.7). Human T cells were stained with anti-human CD3 BUV395 (BD clone SK7), CD4 BV785 (Biolegend clone RPA-T4), and CD8 BV510 (BD clone RPA-T8). Samples were acquired on a BD Fortessa for 60 seconds. Cell counts were normalized to the number of cells entered into the assay. In some conditions, recombinant murine or human CXCL16 (R&D Cat. #503-CX-025 and 976-CX-025, respectively) were used at stated concentrations.

### Example 25

Human CXCL16 ELISA

For in vitro characterization, relevant strains were grown overnight as described above and then sub-cultured (1:100 dilution) for 3 hours. OD₆₀₀ of the cultures were taken, the cultures were then centrifuged (20,817×g for 10′ at 4° C.) and supernatant was entered into a hCXCL16 ELISA (R&D Human CXCL16 DuoSet, Cat. #DY1164). The ELISA was performed as per the manufacturer's protocol. For ELISA on tumor homogenate, after A20 tumors were palpable, they were left untreated or treated twice (3 days apart) as described below with E. coli Nissle expressing wildtype hCXCL16 with or without SLC. Three days after the last treatment, tumors were harvested, weighed, and homogenized in tissue lysis buffer (*** in water) with protease inhibitor and EDTA. The homogenate was then centrifuged (5000×g for 10′ at 4° C.) and the supernatant was used for the ELISA as per the manufacturer's protocol.

### Example 26

Mouse Tumor Cell Line Models

A20 cells were maintained in RPMI supplemented with 10% FBS, Pen/Strep and 2-Mercaptoethanol. MC38 and EO771 cells were maintained in DMEM supplemented with 10% FBS, Pen/Strep, non-essential amino acids, Glutamax, HEPES, Sodium Pyruvate and 2-Mercaptoethanol. Cultures were maintained in a humidified 37° C. 5% CO₂ incubator. Prior to injection, A20 cells were resuspended in in RPMI without phenol red at 5×10⁷ cells/mL. A20 cells were implanted at 100 μL (5×10⁶ cells) per hind flank. MC38 and EO771 cells were washed in PBS, resuspended at 5×10⁶ cells/mL and 10×10⁶ cells/mL in PBS, respectively, and 100 μL of cell suspension was injected subcutaneously into both hind flanks. Female 7-8 week old BALB/c (for A20 tumors) or C57BL/6N (EO771 and MC38 tumors) mice were purchased from Taconic Biosciences or Jackson Laboratories, allowed to acclimate for a week and then injected with tumor cells. A20 tumor volume was determined by caliper measurements (length×width²×0.5) and mice were assigned treatment groups after tumors reached a volume of 100-300 mm³. MC38 and EO771 tumor volume was calculated as length×width×height, and mice were assigned treatment groups after tumors reached a volume of 50-150 mm³. For treatment with bacteria, bacteria were cultured in a 37° C. shaking incubator for up to 12 hours to reach stationary phase of growth in LB broth with appropriate antibiotics and 0.2% glucose. Bacteria were then sub-cultured (1:100 dilution) until a maximum OD₆₀₀ of 0.15 was reached, again in LB broth with appropriate antibiotics and 0.2% glucose, then washed 3× in ice cold PBS and resuspended at a concentration of 1.25×10⁸ or 2.5×10⁷ bacteria/mL to inject 5×10⁶ (A20 tumors) or 1×10⁶ bacteria (MC38 and EO771 tumors) per 40 μL of PBS. Tumors were injected with 20-40 μL of bacteria suspension every 3-4 days for a total of 3-4 treatments as indicated. Mice were euthanized when tumors reached a volume of 1000 mm³ or upon veterinarian recommendation. All animal studies were performed with approved by the Columbia University Institutional Animal Care and Use Committee.

### Example 27

Immune Phenotyping by Flow Cytometry

Tumors were treated as above and harvested at indicated time points. Tumors were harvested, then minced and digested in wash media (RPMI 1640 supplemented with 5% FCS, HEPES, Glutamax, Pen/Strep) with 1 mg/mL collagenase A and 0.5 μg/mL DNAse I in a shaking incubator for up to 45 minutes to achieve a single cell suspension. Once a single cell suspension was achieved, samples were either restimulated or stained for flow cytometry analysis. For cytokine staining and ex vivo restimulation with PMA/ionomycin, aliquots of tumor homogenates were incubated for 3 hours at 37° C. in 10% complete RMPI (as above) with PMA (50 ng/mL), ionomycin (500 ng/mL) and brefeldin A (1 μg/mL) prior to flow cytometry staining. For cytokine staining and ex vivo restimulation with A20 idiotype peptide, aliquots of tumor homogenates were incubated for 5 hours at 37° C. in 10% complete RMPI (as above) with the A20 idiotype peptide (DYWGQGTEL; 1 μg/mL) and brefeldin A (1 μg/mL) prior to flow cytometry staining. Live/dead staining was performed via Ghost Dye Red 780 labeling (Tonbo Biosciences), as per the manufacturer's protocol. Cells were then stained for flow cytometry, with intracellular staining performed using the Tonbo Foxp3/Transcription Factor Staining Buffer Kit as per the manufacturer's protocol. Antibodies used included anti-CD45 (clone 30-F11, Biolegend), NK1.1 (clone PD136, BD Biosciences), CD3e (clone 145-2C11, Tonbo), TCRß (clone H57-597, BD Biosciences), CD4 (clone RM4-5, BD Biosciences), CD8 (clone 53-6.7, Tonbo), Foxp3 (clone FJK-16s, eBioscience), Granzyme-B (clone QA16A02, Biolegend), Ki-67 (clone SolA15, Thermo), IFNγ (clone XMG1.2, Tonbo), B220 (clone RA3-6B2, BD), CD11c (clone N418, Tonbo), Ly6G (clone 1A8, Tonbo), CD11b (clone M1/70, Tonbo), MHCII (clone M5/114.15.2, Tonbo).

### Example 28

Generation and Characterization of CXCL16-Variant Strains in Probiotic E. coli

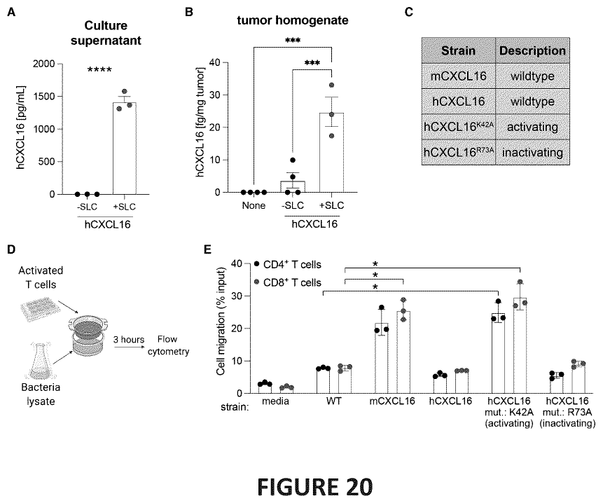

The utility of CXCL16 and variants thereof was further examined. The chemokine CXCL16 recruits specifically memory T cells with extra-lymphoid homing potential, and its expression is associated with improved T cell infiltration and survival in colon and lung cancers, among other cancers. Human CXCL16 (hCXCL16) was expressed on a high copy plasmid in EcN and hCXCL16 was SLC-dependent (FIG. 20A). Release of hCXCL16 in tumors in vivo also was SLC-dependent, with minimal detection in tumors left untreated or treated with EcN expressing hCXCL16 without SLC, but significantly greater detection when SLC was co-expressed (FIG. 20B). To identify the optimal variant of CXCL16, murine CXCL16 (mCXCL16, SEQ ID NO: 12), an activating form of human CXCL16 (hCXCL16^{K42A}) and inactivating form of human CXCL16 (hCXCL16^{R73A}, SEQ ID NO: 13) mutants of human CXCL16 (FIG. 20C) were created.

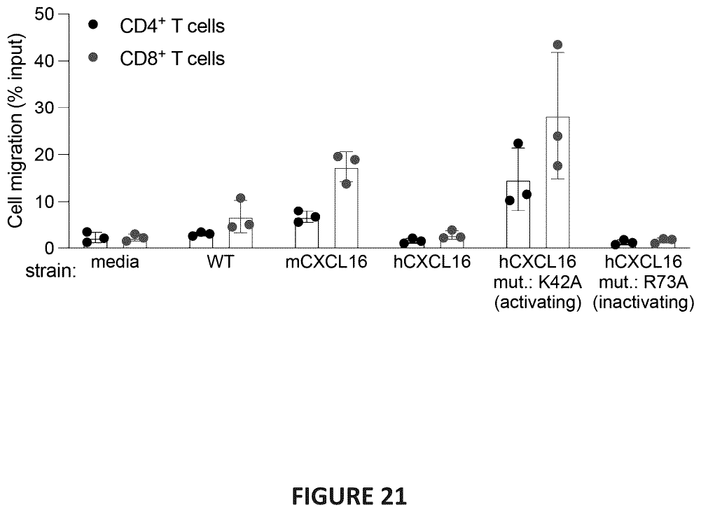

For functional assessment of the probiotic-derived CXCL16 variants, a chemotaxis assay was developed in which activated T cells were assayed for their migration in response to lysate of the EcN strains (FIG. 20D). Compared to wild-type lysate, activated mouse CD4⁺ and CD8⁺ T cells significantly migrated in response to lysate of the activating hCXCL16^{K42A} strain but not wild-type hCXCL16 or inactivating hCXCL16^{R73A} (FIG. 20E). Furthermore, hCXCL16^{K42A} demonstrated similar bioactivity to wild-type mCXCL16 (FIG. 20E). Consistent with previous characterization of the hCXCL16^{K42A} mutation, activated human T cells displayed similar trends in response to lysate of the hCXCL16^{K42A} strain (FIG. 21). These data suggest that E. coli-derived hCXCL16^{K42A} potently attracts mouse and human activated T cells.

### Example 29

Probiotic E. coli-Derived CXCL16 Promotes Mouse Tumor Regression

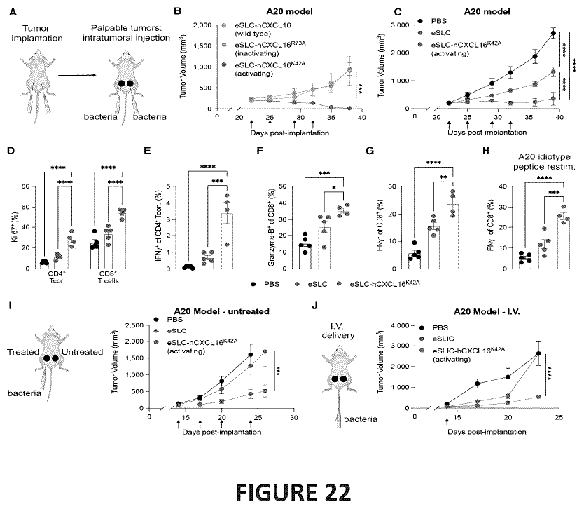

The efficacy of EcN-derived hCXCL16K42A was assessed in vivo by treating subcutaneous murine tumors after they were established and palpable (˜100 mm³). Variants of hCXCL16 were first tested in the A20 B cell lymphoma model, and intratumoral injections of bacteria were performed every 3-4 days for four total treatments (FIG. 22A). Tumor growth was significantly slowed in mice treated with EcN co-expressing SLC and activating hCXCL16^{K42A}(eSLC-hCXCL16^{K42A}) compared to wild-type hCXCL16 (eSLC-hCXCL16) and inactivating mutant hCXCL16^{R73A} (eSLC-hCXCL16^{R73A}) strains (FIGS. 22B & 23A), consistent with the in vitro chemotaxis assay (FIG. 20G) and demonstrating therapeutic activity of hCXCL16^{K42A} in vivo. Furthermore, the hCXCL16^{K42A} strain was significantly more effective than PBS or EcN expressing SLC alone (eSLC) in treating established A20 tumors (FIGS. 22C & 23B). Notably, the hCXCL16^{K42A} strain induced complete regression of 7 of 10 treated A20 tumors. Phenotyping of tumor infiltrating lymphocytes revealed that the hCXCL16^{K42A} strain induced an increase in activated and proliferating CD4⁺ T_{con} cells, as assessed by Ki-67 expression and cytokine production (FIGS. 22D, 22E, & 23C). Treatment with the hCXCL16^{K42A} strain also led to increased CD8⁺ T cell activation in A20 tumors, as assessed by Ki-67 and Granzyme-B expression (FIGS. 22D & 22F), and cytokine production (FIGS. 22G & 23 D). Moreover, upon ex vivo restimulation with an MHC-I (H-2K^{d})-restricted A20 idiotype peptide, CD8⁺ T cells from A20 tumors treated with the hCXCL16^{K42A} strain demonstrated increased cytokine production (FIGS. 22H & 23E), suggesting increased effector function of tumor antigen-specific T cells with hCXCL16^{K42A} treatment. Without wishing to be bound by theory, these data suggest that the hCXCL16^{K42A} strain promotes A20 tumor regression via an expansion of activated T cells, specifically tumor antigen-specific T cells.

In the A20 B cell lymphoma model, treatment with the hCXCL16^{K42A} strain in one tumor led to slowed tumor growth in distant untreated tumors compared to eSLC alone or PBS (FIGS. 22I & 23F), a so-called ‘abscopal effect’. To move beyond injectable tumors, intravenous injection offers a delivery approach for the many tumors that cannot be directly manipulated, as bacteria colonize tumors specifically. EcN hCXCL16^{K42A} (eSLIC-hCXCL16^{K42A}) significantly slowed A20 tumor growth following a single intravenous injection compared to PBS or SLC alone (eSLIC) alone (FIGS. 22I & 23G). Taken together, these data demonstrate that the hCXCL16^{K42A} strain slows tumor growth, including distant tumors left untreated and those treated via intravenous injection, and promotes an expansion of activated CD8⁺ T cells.

### Example 30

Therapeutic Efficacy of CXCL16 in Murine Colorectal Cancer and Breast Cancer

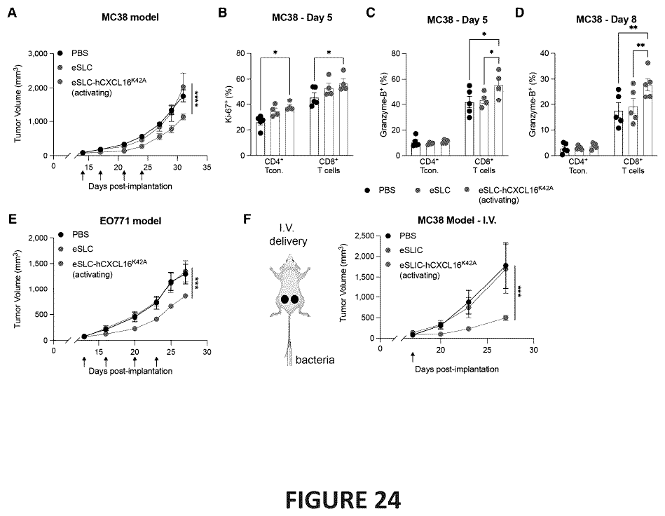

To assess the broader applicability of this approach, the therapeutic efficacy of the hCXCL16^{K42A} strain was examined in more aggressive murine cancer models. Treatment of established MC38 colorectal tumors with the hCXCL16^{K42A} strain slowed tumor growth compared to PBS and eSLC alone (FIGS. 24A & 25A). In this colorectal cancer model, we observed an expansion of proliferating conventional CD4⁺ and CD8⁺ T cells following treatment with the hCXCL16^{K42A} strain at day 5 post-initial treatment (FIG. 24B). Furthermore, treatment with the hCXCL16^{K42A} strain led to an expansion of Granzyme-B⁺CD8⁺ T cells at days 5 and 8 post-initial treatment (FIGS. 24C-D). Treatment with the hCXCL16^{K42A} strain also significantly slowed tumor growth in the TNBC EO771 model compared to PBS or the eSLC strain (FIGS. 24E & 25B). To examine more translational approaches, we again explored the therapeutic efficacy of intravenous delivery. Treatment with the hCXCL16^{K42A} strain significantly slowed MC38 tumor growth after a single intravenous injection compared to PBS or SLC alone treatment (FIGS. 24F & 25C). These data show that the hCXCL16^{K42A} strain offers therapeutic efficacy, including with a single intravenous treatment, and leads to an increase of activated T cells in multiple different murine cancer models, with a more modest benefit in these colorectal and breast cancer models than B cell lymphoma.

### Example 31

Recruitment of Dendritic Cells Synergizes with Activated T Cell Recruitment

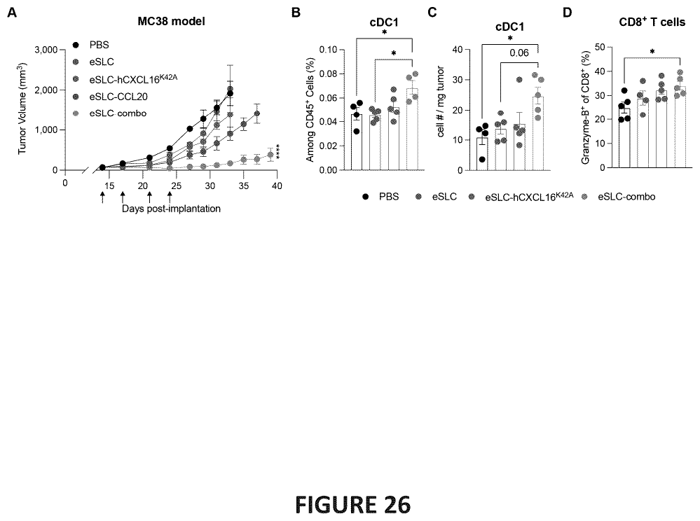

Additional approaches to augment the immune response observed with the hCXCL16^{K42A} strain were also examined. The chemokine CCL20 recruits pre-dendritic cells and, when expressed by tumor cells, demonstrates therapeutic potential and dendritic cell recruitment in vivo. The combination of hCXCL16^{K42A} and CCL20 (SEQ ID NO: 14) strains had a synergistic effect in slowing MC38 tumor growth, slowing tumor growth significantly compared to PBS and eSLC alone, as well as compared to each individual strain (FIGS. 26A & 27). The combination of the hCXCL16^{K42A} and CCL20 strains promoted an expansion by frequency and number of type 1 conventional dendritic cells soon after the initial treatment (FIGS. 26B-C). Finally, this combination led to an expansion of Granzyme-B expressing CD8⁺ T cells (FIG. 26D), consistent with increased activation of CD8⁺ T cells by cDC1s.

While this invention has been disclosed with reference to particular embodiments, it is apparent that other embodiments and variations of the inventions disclosed herein can be devised by others skilled in the art without departing from the true spirit and scope thereof. The appended claims include all such embodiments and equivalent variations.

What is claimed is:

1. A system comprising a first component comprising a programmable bacteria cell and a second component comprising an immune T cell expressing a chimeric antigen receptor (CAR-T cell),
wherein the programmable bacteria cell comprises a synchronized lysis circuit and a nucleic acid sequence that encodes an antigen and is capable of delivering the antigen to a tumor; and
wherein the CAR-T cell is engineered to recognize and respond to the antigen and activate an immune response against the tumor.

2. The system of claim 1, wherein the synchronized lysis circuit comprises a nucleic acid encoding a quorum-sensing gene, a nucleic acid encoding a lysis gene, a promoter, and a terminator contained on a single operon.

3. The system of claim 1 or claim 2, wherein the antigen is synthetic.

4. The system of claim 3, wherein the antigen is a form of green fluorescent protein (GFP).

5. The system of claim 3, wherein the antigen is a form of super-folding green fluorescent protein (sfGFP).

6. The system of claim 5, wherein the antigen has an amino acid sequence set forth as SEQ ID NO: 1 or SEQ ID NO: 2.

7. The system of claim 3, wherein the antigen is a form of ALFA tag protein.

8. The system of claim 7, wherein the antigen has an amino acid sequence set forth as SEQ ID NO: 3.

9. The system of any one of claims 1-8, wherein the programmable bacteria cell further comprises a nucleic acid encoding a cytokine.

10. The system of claim 9, wherein the cytokine is selected from the group consisting of IL-12, CXCL9, CXCL16, or CCL 20.

11. The system of claim 9, wherein the programmable bacteria cell comprises a first nucleic acid encoding a mutant form of CXCL16 having an amino acid sequence set forth as SEQ ID NO: 11 and a second nucleic acid encoding a form of CCL 20 having an amino acid sequence set forth as SEQ ID NO: 14.

12. The system of any one of claims 1-11, wherein the programmable bacterial cells belong to at least one genus selected from the group consisting of Salmonella, Escherichia, Firmicutes, Bacteroidetes, Lactobacillus, and Bifidobacteria.

13. The system of claim 12, wherein the programmable bacterial cells belong to the genus Escherichia.

14. The system of claim 13, wherein the programmable bacterial cells are Escherichia coli Nissle (EcN) cells.

15. The system of claim 14, wherein the EcN cells comprise a knockout of the FliC gene, a knockout of the msbB gene, or a knockout of the FliC gene and the msbB gene.

16. A system comprising a first component comprising a first programmable bacteria cell and a second programmable bacteria cell, and a second component comprising an immune T cell expressing a chimeric antigen receptor (CAR-T cell),
wherein the first programmable bacteria cell comprises a synchronized lysis circuit and a nucleic acid sequence that encodes for a first cytokine;
wherein the second programmable bacteria cell comprises a synchronized lysis circuit and a nucleic acid sequence that encodes that encodes for a second cytokine;
wherein one or both of the first and second programmable bacteria cell comprises a nucleic acid sequence that encodes for an antigen, and wherein both the first and second programmable bacteria cell are capable of delivering each cytokine and the antigen to a tumor; and
wherein the CAR-T cell is engineered to recognize and respond to at least one of the first and second cytokine and is engineered to recognize and respond to the antigen and activate an immune response against the tumor.

17. The system of claim 16, wherein the synchronized lysis circuit comprises a nucleic acid encoding a quorum-sensing gene, a nucleic acid encoding a lysis gene, a promoter, and a terminator contained on a single operon.

18. The system of claim 16 or claim 17, wherein the antigen is synthetic.

19. The system of claim 18, wherein the antigen is a form of green fluorescent protein (GFP).

20. The system of claim 18, wherein the antigen is a form of super-folding green fluorescent protein (sfGFP).

21. The system of claim 20, wherein the antigen has an amino acid sequence set forth as SEQ ID NO: 1 or SEQ ID NO: 2.

22. The system of claim 18, wherein the antigen is a form of ALFA tag protein.

23. The system of claim 22, wherein the antigen has an amino acid sequence set forth as SEQ ID NO: 3.

24. The system of any one of claims 16-23, wherein the first cytokine and the second cytokine are different cytokines and are selected from the group consisting of IL-12, CXCL9, CXCL16, or CCL 20.

25. The system of any one of claims 16-23, wherein the first cytokine is a mutant form of CXCL16 having an amino acid sequence set forth as SEQ ID NO: 11 and the second cytokine is a form of CCL 20 having an amino acid sequence set forth as SEQ ID NO: 14.

26. The system of any one of claims 16-25, wherein the programmable bacterial cells belong to at least one genus selected from the group consisting of Salmonella, Escherichia, Firmicutes, Bacteroidetes, Lactobacillus, and Bifidobacteria.

27. The system of claim 26, wherein the programmable bacterial cells belong to the genus Escherichia.

28. The system of claim 27, wherein the programmable bacterial cells are Escherichia coli Nissle (EcN) cells.

29. The system of claim 28, wherein the EcN cells comprise a knockout of the FliC gene, a knockout of the msbB gene, or a knockout of the FliC gene and the msbB gene.

30. The system of any one of claims 1-15, wherein at least one of the programmable bacterial cell and the CAR-T cell are formulated as a pharmaceutical composition that further comprises a pharmaceutically acceptable carrier, diluent, or excipient.

31. The system of any one of claims 16-29, wherein at least one of the first programmable bacterial cell, the second programmable bacterial cell, and the CAR-T cell are formulated as a pharmaceutical composition that further comprises a pharmaceutically acceptable carrier, diluent, or excipient.

32. A method of treating a hyperproliferative disorder comprising administering to a subject in need thereof a therapeutically effective dose of the system of any one of claims 1-31.

33. The method of claim 32, wherein the hyperproliferative disorder is selected from the group consisting of breast cancer, melanoma, renal cancer, prostate cancer, pancreatic adenocarcinoma, colorectal cancer, lung cancer, esophageal cancer, squamous cell carcinoma of the head and neck, liver cancer, ovarian cancer, cervical cancer, thyroid cancer, glioblastoma, and glioma.

34. A method of reducing the rate of proliferation of a tumor cell comprising delivering the system of any one of claims 1-31 to the tumor cell.

35. A method of killing a tumor cell comprising delivering the system of any one of claims 1-31 to the tumor cell.

36. An article of manufacture useful for treating a hyperproliferative disorder comprising:
a container comprising at least one of the first and second components of the system of any one of claims 1-31; and
instructional materials for using the at least one of the first and second components of the system to treat the hyperproliferative disorder.
